(19) United States
(12) Patent Application Publication
(10) Pub. No.: US 2002/0019389 A1
(43) Pub. Date: Feb. 14, 2002

(54) Urea derivative useful as an anti-cancer agent and process for preparing same

(75) Inventors: Joong Young Kim, Seoul (KR); Byung Hoon Yoon, Seoul (KR); Sun Kyung Hwang, Seoul (KR); Chul Min Oh, Ansan-si (KR); Mee Seon Park, Seoul (KR); Kyoung Ok Song, Seoul (KR); Seong Soo Oh, Seoul (KR)

Correspondence Address: Peter F. Corless, Dike, Bronstein, Roberts & Cushman, IP Group of, EDWARDS & ANGELL, LLP, 130 Water Street, Boston, MA, 02109, US

(73) Assignee: Chaconne Nsi Co., Ltd., Seoul (KR)

(21) Appl. No.: 09/880,823

(22) Filed: Jun. 14, 2001

(30) Foreign Application Priority Data
Jun. 15, 2000 (KR) 2000-32925
Jun. 15, 2000 (KR) 2000-32927
Jun. 15, 2000 (KR) 2000-32930
Aug. 5, 2000 (KR) 2000-45427

(51) Int. Cl. A61K 31/55; A61K 31/551; A61K 31/495; C 07D 4 3/02

(52) U.S. Cl. 514/213.01; 514/217.01; 514/221; 514/254.05; 544/370; 540/569; 540/594

(57) ABSTRACT

The present invention relates to a novel urea derivative represented by the following formula (I), which is useful as an anti-cancer agent:

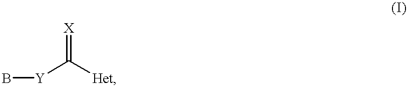

its pharmaceutically acceptable acid addition salt or stereoisomer, in which X, Y, B and Het have the meaning as defined in the specification, and to a process for preparing the compound of formula (I) and an anti-cancer composition comprising the compound of formula (I) as an active ingredient.

### TECHNICAL FIELD

The present invention relates to a novel urea derivative represented by the following formula (I), which is useful as an anti-cancer agent:

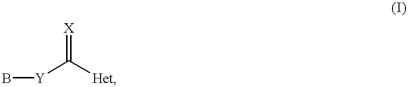

, its pharmaceutically acceptable acid addition salt or stereoisomer, in which

X represents O or S, or represents imino substituted or unsubstituted by cyano,

Y represents a direct bond, NH, O or S,

B represents C₁-C₈-alkyl, or represents a radical having one of the following formulas:

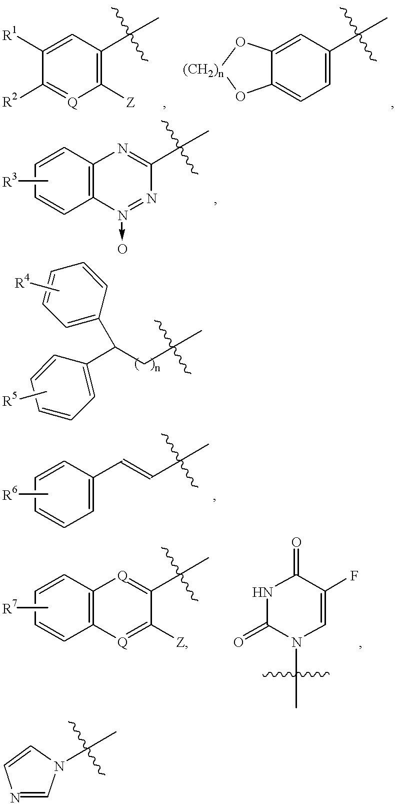

wherein

R¹ and R² independently of one another represent hydrogen, C₁-C₈-alkyl or cyano, or represent amidino substituted or unsubstituted by C₁-C₈-alkyl,

Q represents CH or N,

Z represents C₁-C₄-alkoxy or phenoxy,

n represents an integer of 0 to 3,

R³, R⁴, R⁵, R⁶ and R⁷ independently of one another represent hydrogen, C₁-C₈-alkyl or halogen,

Het represents a radical having one of the following formulas:

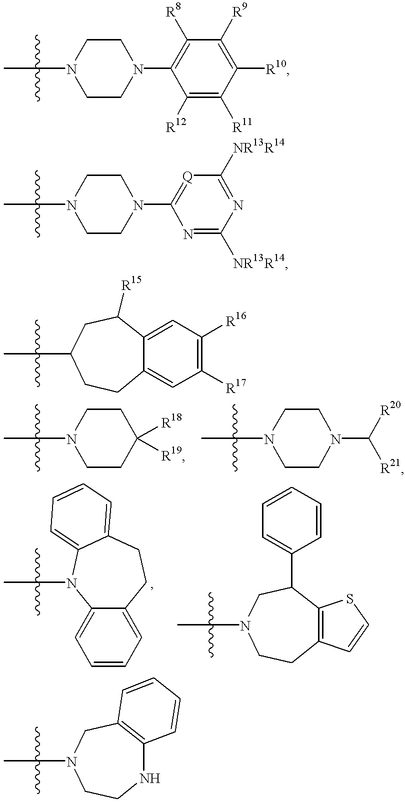

wherein

R⁸, R⁹, R¹⁰, R¹¹ and R¹² independently of one another represent hydrogen, C₁-C₈-alkyl, C₁-C₈-alkoxy or halogen,

R¹³ and R¹⁴ independently of one another represent hydrogen, C₁-C₈-alkyl, C₁-C₈-alkoxy, C₂-C₅-alkenyl, C₃-C₆-cycloalkyl or C₃-C₆-cycloalkyl-C₁-C₄- alkyl, or R¹³ and R¹⁴ together with the nitrogen atom to which they are attached represent pyrrolinyl or pyrrolidinyl,

R¹⁵ represents hydrogen, or represents phenyl or benzyl each of which is substituted or unsubstituted by 1 to 5 identical or different halogen atoms,

R¹⁶ and R¹⁷ independently of one another represent hydrogen, C₁-C₄-alkyl, C₁-C₄-alkoxy or hydroxy,

R¹⁸ and R¹⁹ independently of one another represent hydrogen, hydroxycarbonyl or C₁-C₄-alkoxycarbonyl, or represent phenyl substituted or unsubstituted by 1 to 5 identical or different halogen atoms or C₁-C₄-alkoxy, or together represent diphenylmethylene or benzolactone of formula

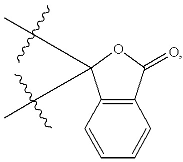

and

R²⁰ and R²¹ independently of one another represent hydrogen, or represent phenyl substituted or unsubstituted by 1 to 5 identical or different halogen atoms, C₁-C₈-alkyl or C₁-C₄-alkoxy, or represent C₁-C₄-alkoxycarbonyl.

The present invention also relates to a process for preparing the compound of formula (I), and an anti-cancer composition comprising the compound of formula (I) as an active ingredient.

### BACKGROUND ART

As anti-cancer agents, cisplatin, doxorubicin (adriamycin), 5-FU, camptothecin, taxol, etc. are currently known and used clinically. One of platinum complex as anti-cancer agents, cisplatin, shows its activity based on the DNA alkylation reaction. But, it is not freed from severe side effects and toxicity due to the heavy metal. Further, as anti-cancer agents such as doxorubicin (adriamycin), etc., which show their activity through the intercalation mechanism, have lower selectivity, they could also lead to these adverse and undesirable effects. Meanwhile, the taxol derivative, which has been recently developed and proved an effective weapon against breast, ovarian, lung cancer, etc., requires a special formulation because it is sparingly soluble in water. Therefore, severe side effects may also be caused by the use of the excipients required for the formulation.

For these reasons, it has been needed to develop a new anti-cancer agent which can realize a superior anti-cancer activity as well as overcome the problems (low selectivity against the solid tumors in human body, low solubility in water, side effects, toxicity, etc.) aligned to the existing agents.

### DISCLOSURE OF INVENTION

Thus, the present inventors have extensively studied to minimize the problems of the existing anti-cancer agents, and to develop a new compound having a potent and effective anti-cancer activity against the solid tumors in human body. As a result, we have identified that the urea derivative of formula (I) above meets the requirements of potent anti-cancer activity and low toxicity, and then completed the present invention.

Therefore, the object of the present invention is to provide the urea derivative of formula (I), as defined above, its pharmaceutically acceptable salt and stereoisomer.

It is another object of the present invention to provide a process for preparing the compound of formula (I).

It is still another object of the present invention to provide an anti-cancer composition having a superior physiological activity against solid cancers in human body and a low toxicity comprising the compound of formula (I) as an active-ingredient together with a pharmaceutically acceptable carrier.

### BEST MODE FOR CARRYING OUT THE INVENTION

The present invention relates to a novel urea derivative represented by the following formula (I), which shows a superior anti-cancer activity and a low toxicity:

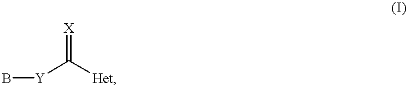

, its pharmaceutically acceptable acid addition salt or stereoisomer, in which

X represents O or S, or represents imino substituted or unsubstituted by cyano,

Y represents a direct bond, NH, O or S,

B represents C₁-C₈-alkyl, or represents a radical having one of the following formulas:

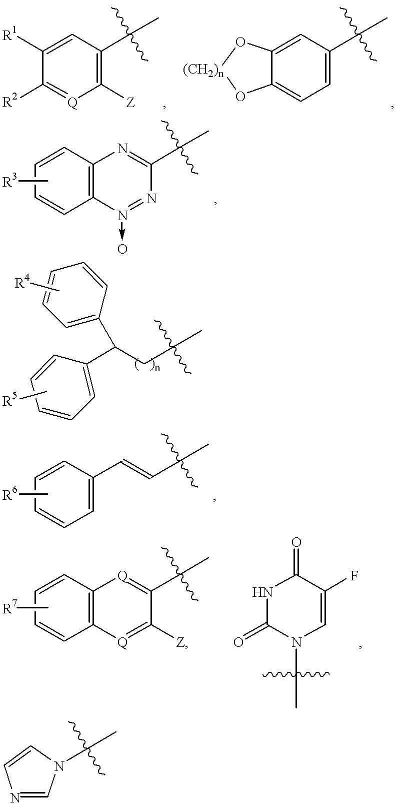

wherein

R¹ and R² independently of one another represent hydrogen, C₁-C₈-alkyl or cyano, or represent amidino substituted or unsubstituted by C₁-C₈-alkyl,

Q represents CH or N,

Z represents C₁-C₄-alkoxy or phenoxy,

n represents an integer of 0 to 3,

R³, R⁴, R⁵, R⁶ and R⁷ independently of one another represent hydrogen, C₁-C₈-alkyl or halogen,

Het represents a radical having one of the following formulas:

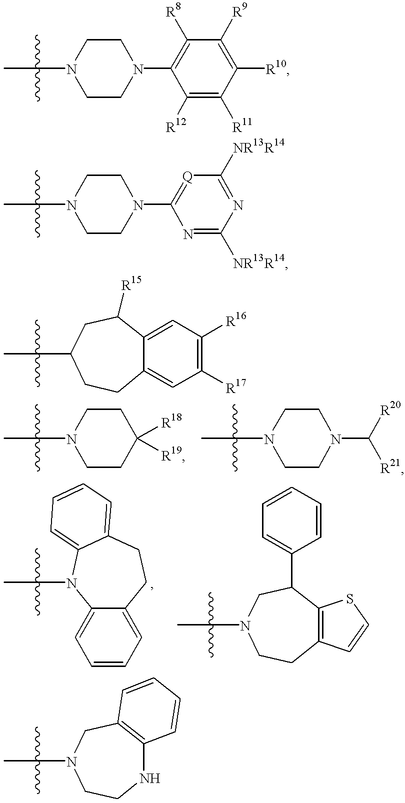

wherein

R⁸, R⁹, R¹⁰, R¹¹ and R¹² independently of one another represent hydrogen, C₁-C₈-alkyl, C₁-C₈-alkoxy or halogen,

R¹³ and R¹⁴ independently of one another represent hydrogen, C₁-C₈-alkyl, C₁-C₈-alkoxy, C₂-C₅-alkenyl, C₃-C₆-cycloalkyl or C₃-C₆-cycloalkyl-C₁-C₄- alkyl, or R¹³ and R¹⁴ together with the nitrogen atom to which they are attached represent pyrrolinyl or pyrrolidinyl,

R¹⁵ represents hydrogen, or represents phenyl or benzyl each of which is substituted or unsubstituted by 1 to 5 identical or different halogen atoms,

R¹⁶ and R¹⁷ independently of one another represent hydrogen, C₁-C₄-alkyl, C₁-C₄-alkoxy or hydroxy,

R¹⁸ and R¹⁹ independently of one another represent hydrogen, hydroxycarbonyl or C₁-C₄-alkoxycarbonyl, or represent phenyl substituted or unsubstituted by 1 to 5 identical or different halogen atoms or C₁-C₄-alkoxy, or together represent diphenylmethylene or benzolactone of formula

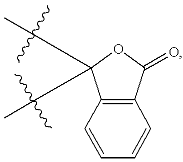

and

R²⁰ and R²¹ independently of one another represent hydrogen, or represent phenyl substituted or unsubstituted by 1 to 5 identical or different halogen atoms, C₁-C₈-alkyl or C₁-C₄-alkoxy, or represent C₁-C₄-alkoxycarbonyl.

In the definitions for the substituents of the compound of formula (I), the term “alkyl” used alone or in a composite term such as “alkoxy” means a straight-chain or branched saturated hydrocarbon radical such as methyl, ethyl, propyl, isopropyl, n-butyl, isobutyl and t-butyl, etc.; the term “alkenyl” means an unsaturated hydrocarbon radical having one or more double bonds therein such as ethenyl, propenyl, butenyl, etc.; the term “cycloalkyl” means cyclopropyl, cyclobutyl, cyclopentyl or cyclohexyl; the term “alkoxycarbonyl” means methoxycarbonyl, ethoxycarbonyl, etc.; and the term “halogen” means fluorine, chlorine, bromine or iodine.

Typical examples of the compound of formula (I) according to the present invention are represented in the following Table 1:

TABLE 1a

| | |
|---|---|
| | (Ia) |
| 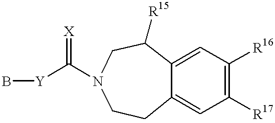 | |

| No. | B | Y | X | R¹⁵ | R¹⁶ | R¹⁷ |
|---|---|---|---|---|---|---|
| 1 | 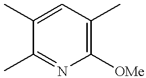 | NH | O | 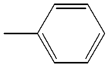 | OMe | OMe |
| 2 | 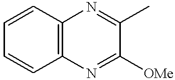 | NH | O | 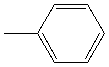 | OMe | OMe |
| 3 | 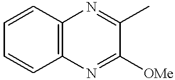 | NH | O | 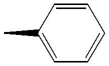 | OMe | OMe |
| 4 | 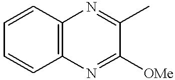 | NH | O | 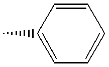 | OMe | OMe |
| 5 | 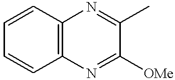 | NH | O | 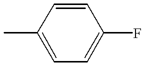 | OMe | OMe |
| 6 | 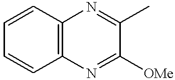 | NH | O | 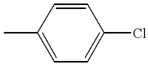 | OMe | OMe |
| 7 | 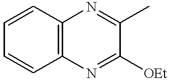 | NH | O | 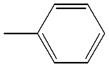 | OMe | OMe |
| 8 | 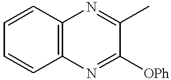 | NH | O | 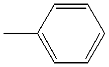 | OMe | OMe |
| 9 | 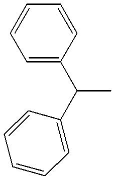 | NH | O | 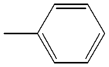 | OMe | OMe |
| 10 | 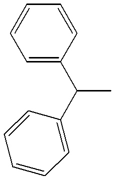 | NH | O | 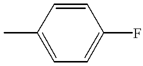 | OMe | OMe |
| 11 | 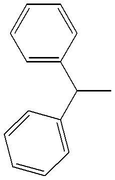 | NH | O | 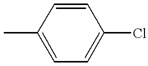 | OMe | OMe |

TABLE 1b

| | |
|---|---|
| | (Ib) |
| 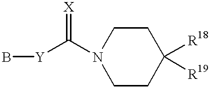 | |

| No. | B | Y | X | R¹⁸ | R¹⁹ |
|---|---|---|---|---|---|
| 12 | 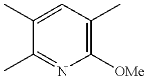 | NH | O | 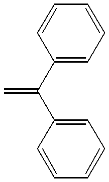 | |
| 13 | 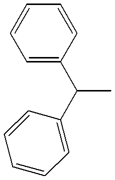 | NH | O | 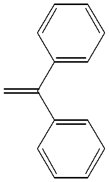 | |
| 14 | 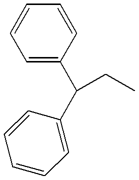 | NH | O | 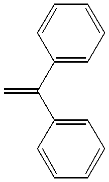 | |
| 15 | 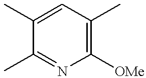 | NH | O | Ph | Ph |
| 16 | 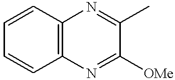 | NH | O | Ph | Ph |
| 17 | 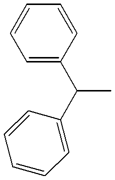 | NH | O | Ph | Ph |
| 18 | 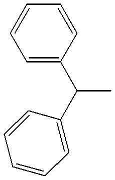 | NH | O | COOH | Ph |
| 19 | 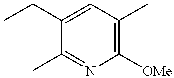 | NH | O | COOH | Ph |
| 20 | 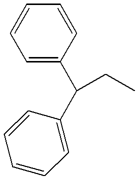 | NH | O | COOH | Ph |

| | | | | |
|---|---|---|---|---|
| 21 | 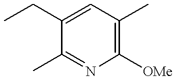 | NH | O | 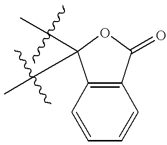 |
| 22 | 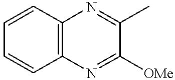 | NH | O | 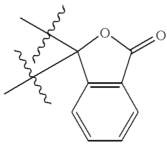 |

TABLE 1c

| | |
|---|---|
| | (Ic) |
| 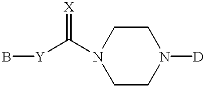 | |

| No. | B | Y | X | D |
|---|---|---|---|---|
| 23 | 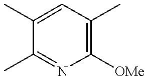 | NH | O | 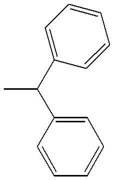 |
| 24 | 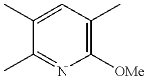 | NH | N—CN | 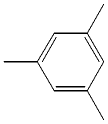 |
| 25 | 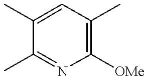 | NH | O | 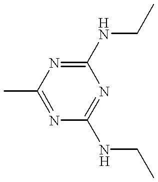 |
| 26 | 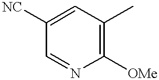 | NH | O | 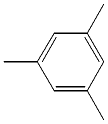 |
| 27 | 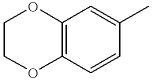 | NH | N—CN | 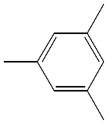 |
| 28 | 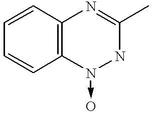 | NH | O | 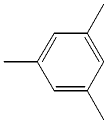 |
| 29 | 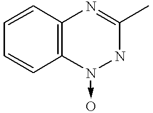 | NH | O | 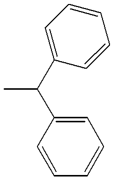 |
| 30 | 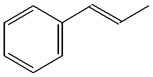 | NH | O | 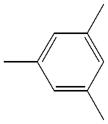 |
| 31 | 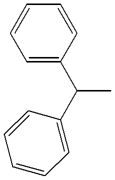 | NH | O | 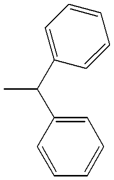 |
| 32 | 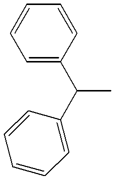 | NH | O | 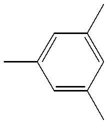 |
| 33 | 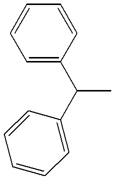 | NH | S | 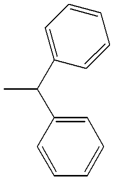 |
| 34 | 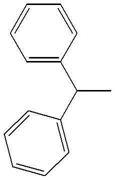 | NH | O | 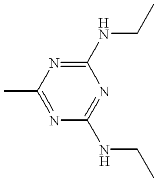 |
| 35 | 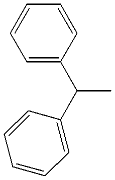 | NH | N—CN | 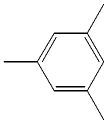 |
| 36 | 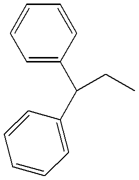 | NH | O | 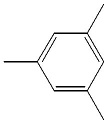 |
| 37 | 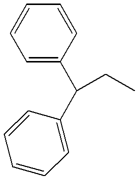 | NH | O | 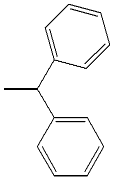 |
| 38 | 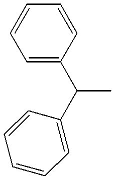 | NH | O | 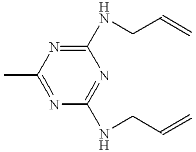 |
| 39 | 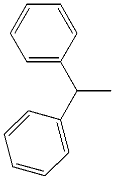 | NH | O | 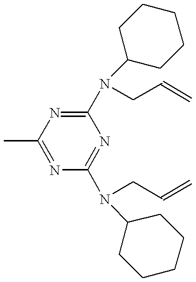 |
| 40 | 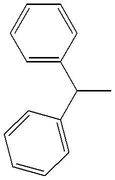 | NH | O | 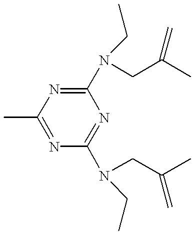 |
| 41 | 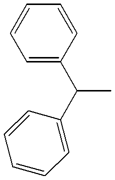 | NH | O | 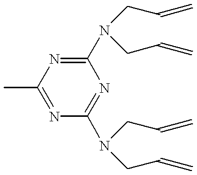 |
| 42 | 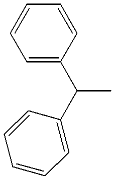 | NH | O | 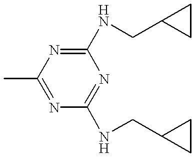 |
| 43 | 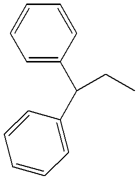 | NH | N—CN | 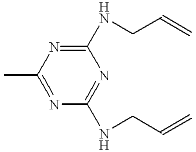 |
| 44 | 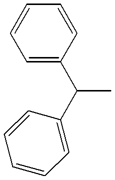 | NH | N—CN | 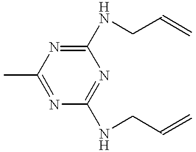 |
| 45 | 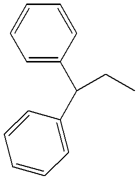 | NH | O | 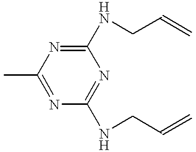 |
| 46 | 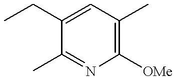 | NH | O | 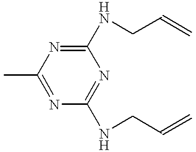 |
| 47 | 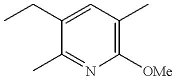 | NH | O | 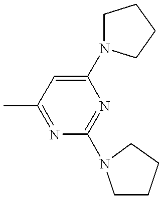 |
| 48 | 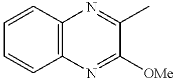 | NH | O | 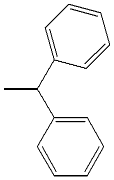 |
| 49 | 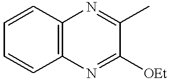 | NH | O | 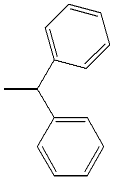 |
| 50 | 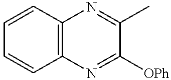 | NH | O | 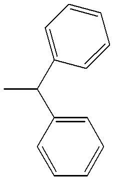 |
| 51 | 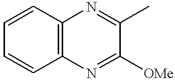 | NH | O | 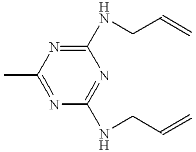 |
| 52 | 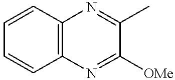 | NH | O | 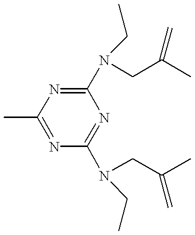 |
| 53 | 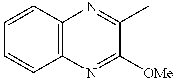 | NH | O | 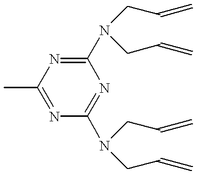 |
| 54 | 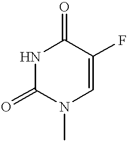 | Direct bond | O | 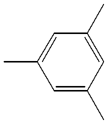 |

TABLE 1d

| | |
|---|---|
| | (Id) |
| 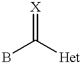 | |

| No. | B | X | Het |
|---|---|---|---|
| 55 | 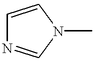 | O | 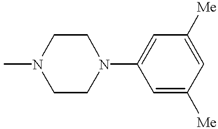 |
| 56 | 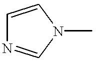 | S | 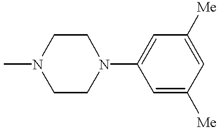 |
| 57 | 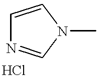 | O | 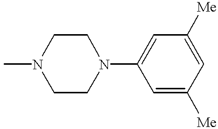 |
| 58 | 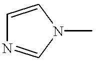 | O | 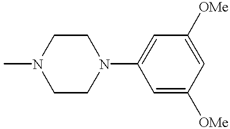 |
| 59 | 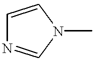 | O | 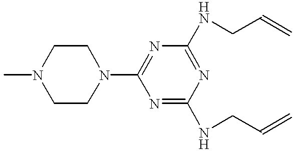 |
| 60 | 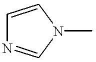 | S | 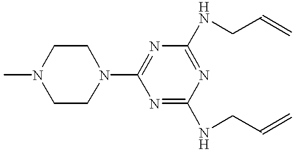 |
| 61 | 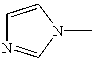 | O | 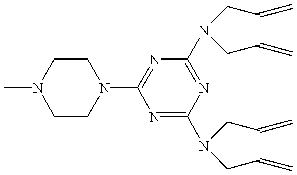 |
| 62 | 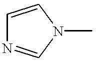 | O | 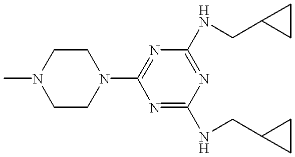 |
| 63 | 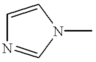 | O | 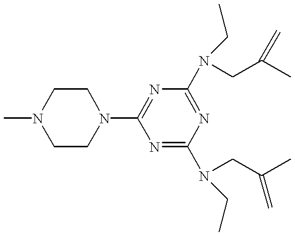 |
| 64 | 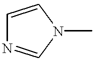 | O | 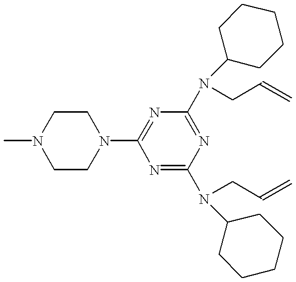 |
| 65 | 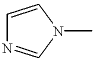 | O | 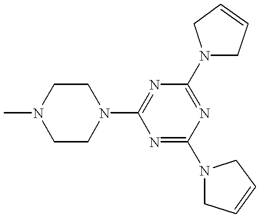 |
| 66 | 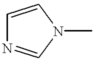 | O | 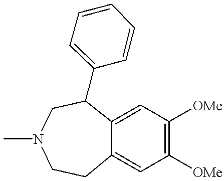 |
| 67 | 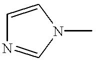 | S | 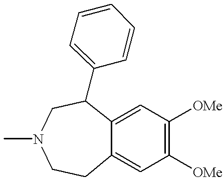 |
| 68 | 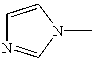 | O | 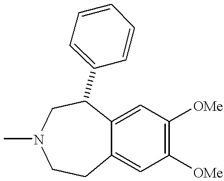 |
| 69 | 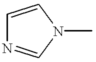 | O | 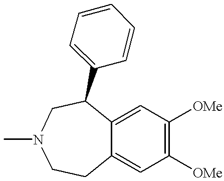 |
| 70 | 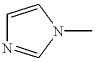 | O | 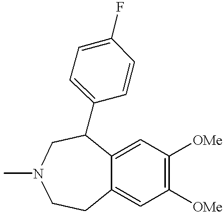 |
| 71 | 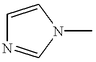 | O | 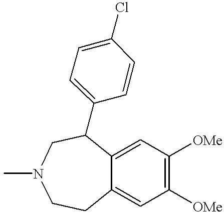 |
| 72 | 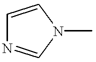 | O | 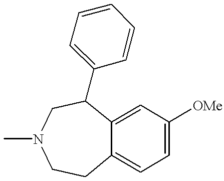 |
| 73 | 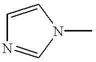 | O | 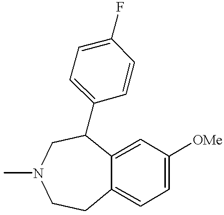 |
| 74 | 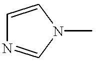 | O | 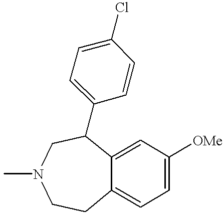 |
| 75 | 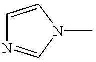 | O | 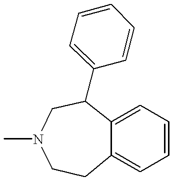 |
| 76 | 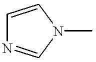 | O | 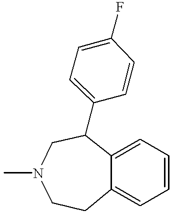 |
| 77 | 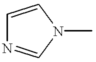 | O | 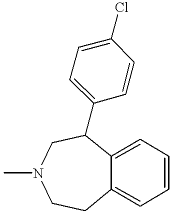 |
| 78 | 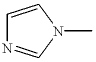 | O | 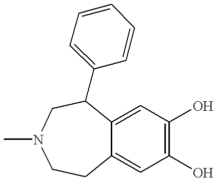 |
| 79 | 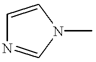 | O | 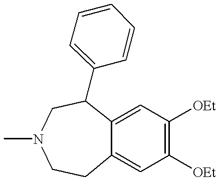 |
| 80 | 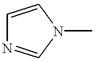 | O | 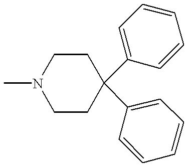 |
| 81 | 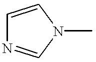 | S | 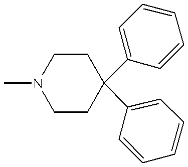 |
| 82 | 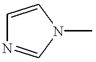 | O | 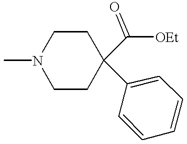 |
| 83 | 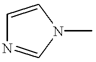 | O | 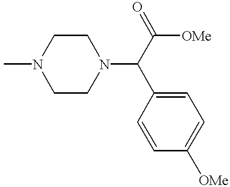 |
| 84 | 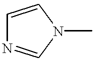 | O | 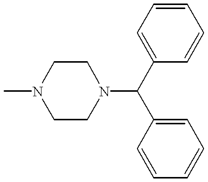 |
| 85 | 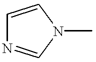 | O | 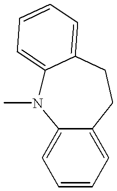 |
| 86 | 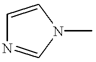 | O | 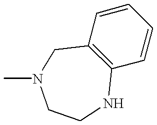 |
| 87 | 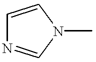 | O | 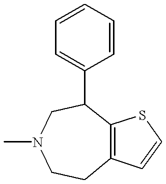 |

Also, the compound of formula (I) according to the present invention can form a pharmaceutically acceptable acid addition salt. Such acid addition salt includes non-toxic acid addition salt containing pharmaceutically acceptable anion, for example a salt with inorganic acids such as hydrochloric acid, sulfuric acid, nitric acid, phosphoric acid, hydrobromic acid, hydriodic acid, etc., a salt with organic carboxylic acids such as tartaric acid, formic acid, citric acid, acetic acid, propionic acid, trichloroacetic acid, trofluoroacetic acid, gluconic acid, benzoic acid, lactic acid, fumaric acid, maleic acid, etc., or a salt with amino acids such as serine, cysteine, cystine, aspartic acid, glutamic acid, lysine, arginine, tyrosine, proline, etc., or a salt with sulfonic acids such as methanesulfonic acid, ethanesulfonic acid, benzenesulfonic acid, p-toluenesulfonic acid, naphthalenesulfonic acid, etc.

Since the compounds of the present invention may have asymmetric carbon center(s) depending on the kind of substituents, they can exist as an enantiomer of R or S, diastereomer, or mixtures thereof including racemate. Therefore, the present invention also includes each of these stereoisomers and their mixtures.

Another object of the present invention is to provide a process for preparing the compound of formula (I) as defined above.

According to the present invention, the compound of formula (I), as defined above, can be prepared by a process characterized in that

(a) a compound represented by the following formula (II):

B—YH (II)

wherein B and Y are as defined above, and a compound represented by the following formula (III):

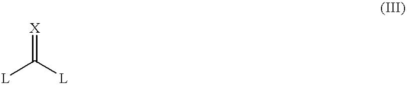

wherein X is as defined above, and L represents a leaving group, preferably C₁-C₄-alkoxy, phenoxy, p-toluenesulfonyl, benzenesulfonyl, p-nitrosulfonyl, halogen or imidazole, are reacted in a solvent in the presence of a base with a compound represented by the following formula (IV):

H—Het (IV)

wherein Het is as defined above, to produce the compound of formula (I); or

(b) a compound represented by the following formula (V):

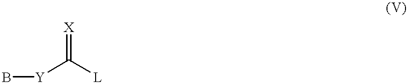

wherein B, Y, X and L are as defined above, is reacted in a solvent in the presence of a base with the compound of formula (IV) to produce the compound of formula (I); or

(c) a compound represented by the following formula (VI):

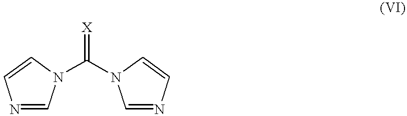

wherein X is as defined above, is reacted in a solvent in the presence of a base with the compound of formula (IV) to produce a compound represented by the following formula (Ida):

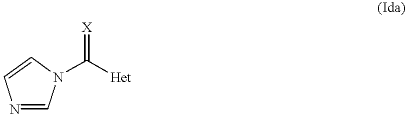

wherein X and Het are as defined above, or optionally deprotection, alkylation or esterification reaction is further carried out.

The above process variants (a) to (c) can be depicted as follows.

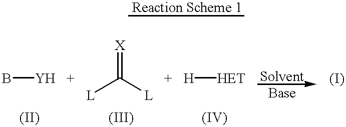

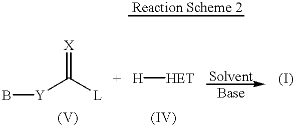

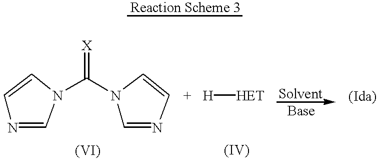

In the process variant (a), the compounds of formulae (II) and (IV) are commercially available, or may be prepared according to the art-known methods (see: J. Med. Chem., 35, 3792(1992), U.S. Pat. No. 4,011,319). Several compounds having the formula (III) have been known and any one selected from phosgene, liquid and solid phosgene, thiophosgene, 1,1′-carbonyldiimidazole, 1,1′-thiocarbonyldiimidazole, diphenylcarbonate, diphenylthiocarbonate, chloromethylformate, chloroethylformate, and chlorophenylformate can be preferably used in the present invention. In order to prepare N-cyanocarbo-imidate, diphenyl N-cyanocarboimidate can be used.

The compound of formula (II) can be preferably used in an amount of 0.5 to 2 equivalents with respect to the compound of formula (IV), and the compound of formula (III) can be preferably used in an amount of 0.5 to 3 equivalents with respect to the same compound of formula (IV).

Any solvent which does not adversely affect to the reaction, preferably one or more selected from a group consisting of methylene chloride, chloroform, tetrahydrofuran, acetonitrile and dimethylformamide, more preferably a solvent having a comparatively high polarity such as tetrahydrofuran or dimethylformamide can be used.

Any conventional organic or inorganic base can be used. The preferable inorganic base includes sodium hydride, sodium hydroxide, potassium hydroxide, sodium carbonate, potassium carbonate, potassium hydrogen carbonate, sodium hydrogen carbonate and cesium carbonate, and the preferable organic base includes trimethylamine, triethylamine, tributylamine, pyridine, dimethylaminopyridine, DBU, and DBN. The base can be used in an amount of 1 to 3 equivalents with respect to the compound of formula (IV).

The reaction is preferably carried out at temperatures ranging from 20 to 100° C.

In process variant (b), the starting compound of formula (V) is commercially available, or may be easily prepared by reacting the compound of formula (II) with the compound of formula (III). The compound of formula (V) can be preferably used in an amount of 0.5 to 2 equivalents with respect to the compound of formula (IV).

The same solvents and bases mentioned for process variant (a) can be used in process variant (b), and the reaction temperature can be selected from the same range.

1,1′-Carbonyldiimidazole or 1,1′-thiocarbonyldiimidazole of formula (VI), used in process variant (c), is commercially available. The compound of formula (VI) can be used in an amount of 0.5 to 2 equivalents with respect to the compound of formula (IV).

The same solvents and bases mentioned for process variant (a) can be used in process variant (c), and the reaction temperature can be selected from the same range. The reaction time may be generally selected from a range of from 0.5 to 48 hours.

The compound of formula (I) prepared according to the process as explained above can be effectively used as an anti-cancer agent as already mentioned. Therefore, the present invention also provides an anti-cancer composition comprising as an active ingredient the compound of formula (I), its pharmaceutically acceptable acid-addition salt, or stereoisomer, together with a pharmaceutically acceptable carrier.

When the active compound according to the present invention is used for clinical purpose, it is preferably administered in an amount ranging from 1 to 1000 mg per kg of body weight a day. The total daily dosage may be administered in one time or over several times. However, the specific administration dosage for the patient can be varied with the specific compound used, body weight of the subject patient, sex, hygienic condition, diet, time or method of administration, excretion rate, mixing ratio of the agent, severity of the disease to be treated, etc.

The compound of the present invention may be administered in the form of injections or oral preparations. Injections, for example, sterilized aqueous or oily suspension for injection, can be prepared according to the known procedure using suitable carriers. Solvents which can be used for preparing injections include water, Ringer's fluid and isotonic NaCl solution, and also sterilized fixing oil may be conveniently used as the solvent or suspending media. Any non-stimulative fixing oil including mono-, di-glyceride may be used for this purpose. Fatty acid such as oleic acid may also be used for injections.

As the solid preparation for oral administration, capsules, tablets, pills, powders and granules, etc., preferably capsules and tablets can be mentioned. It is also desirable for tablets and pills to be formulated into enteric-coated preparation. The solid preparations may be prepared by mixing the active compound of formula (I) according to the present invention with at least one inert carriers.

The pharmaceutically acceptable carriers which can be used for preparing the pharmaceutical composition of the present invention include dispersing agent, wetting agent, suspending agent, lubricant, sweetening agent, binding agent, solubilizer, solubilizing aid, emulsifier, isotonizing agent, adsorbent, disintegrating agent, antioxidant, preservative, glidant, filler, fragrant, etc., more specifically lactose, dextrose, sucrose, starch, mannitol, sorbitol, cellulose, glycine, silica, talc, stearic acid, stearine, tragacanth gum, methyl cellulose, sodium carboxylmethylcellulose, agar, magnesium stearate, alginic acid, water, ethanol, polyethyleneglycol, polyvinylpyrrolidone, sodium chloride, potassium chloride, orange essence, strawberry essence, vanilla, etc.

The present invention, particularly the preparing process and pharmacological effect as explained above, will be more specifically explained in the following examples and experimentals. However, it should be understood that the following examples and experimentals are intended to illustrate the present invention but not in any manner to limit the scope of the present invention.

### EXAMPLE 1

Synthesis of 3-[N-(5,6-dimethyl-2-methoxypyridin-3-yl)aminocarbonyl]-7,8-dimethoxy-1-phenyl-2,3,4,5-tetrahydro-3H-benzazepine

N-(5,6-dimethyl-2-methoxypyridin-3-yl)phenylcarbamate (71 mg, 0.26 mmol) and 7,8-dimethoxy-1-phenyl-2,3,4,5-tetrahydro-3H-benzazepine (67.2 mg, 0.28 mmol) were mixed in anhydrous THF (5 ml), and then DBU (39 μl, 0.26 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 89.6 mg (Yield 81.8%) of the title compound as a solid.

m.p.: 88-90° C.

¹H NMR (300 MHz, CDCl₃): δ ppm 7.14-7.34 (m, 5H), 6.79 (s, 1H), 6.66 (s, 1H), 6.45 (s, 1H), 4.58 (dd, 1H, J=10.06 Hz, J=5.64 Hz), 4.24 (dd, 1H, J=14.94 Hz, J=5.58 Hz), 3.93 (s, 3H), 3.88 (s, 3H), 3.60-3.95 (m, 3H), 3.67 (s, 3H), 3.39 (m, 1H), 2.89 (m, 1H), 2.32 (s, 3H), 2.16 (s, 3H)

### EXAMPLE 2

Synthesis of 3-[N-(2-methoxyquinoxalin-3-yl)aminocarbonyl]-7,8-dimethoxy-1-phenyl-2,3,4,5-tetrahydro-3H-benzazepine

N-(2-methoxyquinoxalin-3-yl)phenylcarbamate (3.4 g, 10.17 mmol) and 7,8-dimethoxy-1-phenyl-2,3,4,5-tetrahydro-3H-benzazepine (3.26 g, 10.17 mmol) were mixed in anhydrous THF (57 ml), and then DBU (1.7 ml, 10.17 mmol) was added dropwise to this reaction solution. After stirring for 1 hour at room temperature, the product was extracted with methylene chloride (200 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 3.69 g (Yield 66.1%) of the title compound as a solid.

m.p.: 96-98° C.

¹H NMR (300 MHz, CDCl₃): δ ppm 6.49-7.83 (m, 12H), 4.60 (m, 1H), 4.30 (m, 1H), 4.11 (s, 3H), 3.88 (m, 5H), 3.70 (m, 4H), 3.30 (m, 1H), 2.86 (m, 1H)

### EXAMPLE 3

Synthesis of 3-[N-(2-methoxyquinoxalin-3-yl)aminocarbonyl]-(S)-7,8-dimethoxy-1-phenyl-2,3,4,5-tetrahydro-3H-benzazepine

N-(2-methoxyquinoxalin-3-yl)phenylcarbamate (112.1 mg, 0.38 mmol) and (S)-7,8-dimethoxy-1-phenyl-2,3,4,5-tetrahydro-3H-benzazepine (107.6 mg, 0.38 mmol) were mixed in anhydrous THF (2 ml), and then DBU (70 μl, 0.47 mmol) was added dropwise to this reaction solution. After stirring for 3 hours at room temperature, the product was extracted with methylene chloride (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:3, v/v) to give 41.3 mg (Yield 24.1%) of the title compound as a solid.

m.p.: 143-144° C.

¹H NMR (500 MHz, CDCl₃) δ ppm 6.49-7.83 (m, 12H), 4.60 (m, 1H), 4.30 (m, 1H), 4.11 (s, 3H), 3.88 (m, 5H), 3.70 (m, 4H), 3.30 (m, 1H), 2.86 (m, 1H)

### EXAMPLE 4

Synthesis of 3-[N-(2-methoxyquinoxalin-3-yl)aminocarbonyl]-(R)-7,8-dimethoxy-1-phenyl-2,3,4,5- tetrahydro-3H-benzazepine

N-(2-methoxyquinoxalin-3-yl)phenylcarbamate (100 mg, 0.34 mmol) and (R)-7,8-dimethoxy-1-phenyl-2,3,4,5-tetrahydro-3H-benzazepine (95.9 mg, 0.34 mmol) were mixed in anhydrous THF (2 ml), and then DBU (70 μl, 0.47 mmol) was added dropwise to this reaction solution. After stirring for 1 hour at room temperature, the product was extracted with methylene chloride (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:3, v/v) to give 124.7 mg (Yield 81.7%) of the title compound as a solid.

m.p.: 92-94° C.

¹H NMR (500 MHz, CDCl₃): δ ppm 6.48-7.82 (m, 12H), 4.60 (m, 1H), 4.30 (m, 1H), 4.11 (s, 3H), 3.88 (m, 5H), 3.70 (m, 4H), 3.30 (m, 1H), 2.86 (m, 1H)

### EXAMPLE 5

Synthesis of 3-[N-(2-methoxyquinoxalin-3-yl)aminocarbonyl]-7,8-dimethoxy-1-(4-fluoro)phenyl-2,3,4,5-tetrahydro-3H-benzazepine

N-(2-methoxyquinoxalin-3-yl)phenylcarbamate (100 mg, 0.34 mmol) and 7,8-dimethoxy-1-(4-fluoro)phenyl-2,3,4,5-tetrahydro-3H-benzazepine (101.7 mg, 0.34 mmol) were mixed in anhydrous THF (2 ml), and then DBU (70 μl, 0.47 mmol) was added dropwise to this reaction solution. After stirring for 1 hour at room temperature, the product was extracted with methylene chloride (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:3, v/v) to give 109.9 mg (Yield 69.2%) of the title compound as a solid.

m.p.: 96-98° C.

¹H NMR (500 MHz, CDCl₃): δ ppm 6.46-7.81 (m, 11H), 4.59 (m, 1H), 4.25 (m, 1H), 4.12 (s, 3H), 3.88 (m, 5H), 3.72 (m, 4H), 3.31 (m, 1H), 3.21 (m, 1H), 2.85 (m, 1H)

### EXAMPLE 6

Synthesis of 3-[N-(2-methoxyquinoxalin-3-yl)aminocarbonyl]-7,8-dimethoxy-1-(4-chloro)phenyl-2,3,4,5-tetrahydro-3H-benzazepine

N-(2-methoxyquinoxalin-3-yl)phenylcarbamate (35.3 mg, 0.12 mmol) and 7,8-dimethoxy-1-(4-chloro)phenyl-2,3,4,5-tetrahydro-3H-benzazepine (38.0 mg, 0.12 mmol) were mixed in anhydrous THF (0.6 ml), and then DBU (20 μl, 0.13 mmol) was added dropwise to this reaction solution. After stirring for 1 hour at room temperature, the product was extracted with methylene chloride (30 ml×3). The organic solution was washed with aqueous sodium chloride solution and water, dried over anhydrous magnesium sulfate, and concentrated under reduced pressure. The residue was separated by column chromatography(eluent:ethyl acetate:hexane=2:3, v/v) to give 20.4 mg (Yield 32.8%) of the title compound as a solid.

m.p. 94-97° C.

¹H NMR (500 MHz, CDCl₃): δ ppm 6.46-7.47 (m, 11H), 4.54 (m, 1H), 4.20 (m, 1H), 4.12 (m, 3H), 3.88 (m, 5H), 3.72 (m, 4H), 3.25 (m, 1H), 2.86 (m, 1H)

### EXAMPLE 7

Synthesis of 3-[N-(2-ethoxyquinoxalin-3-yl)aminocarbonyl]-7,8-dimethoxy-1-phenyl-2,3,4,5-tetrahydro-3H-benzazepine

N-(2-ethoxyquinoxalin-3 -yl)phenylcarbamate (100 mg, 0.32 mmol) and 7,8-dimethoxy-1-phenyl-2,3,4,5-tetrahydro-3H-benzazepine (90.5 mg, 0.32 mmol) were mixed in anhydrous THF (2 ml), and then DBU (70 μl, 0.47 mmol) was added dropwise to this reaction solution. After stirring for 2 hours at room temperature, the product was extracted with methylene chloride (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:3, v/v) to give 109.5 mg (Yield 68.7%) of the title compound as a solid.

m.p.: 136-138° C.

¹H NMR (500 MHz, CDCl₃): δ ppm 6.47-7.80 (m, 12H), 4.65 (m, 1H), 4.58 (q, 2H) 4.35 (m, 1H), 3.88 (m, 4H), 3.78 (m, 2H), 3.70 (s, 3H), 3.30 (m, 1H), 2.88 (m, 1H), 1.46 (t, 3H)

### EXAMPLE 8

Synthesis of 3-[N-(2-phenoxyquinoxalin-3-yl)aminocarbonyl]-7,8-dimethoxy-1-phenyl-2,3,4,5-tetrahydro-3H-benzazepine

N-(2-phenoxyquinoxalin-3-yl)phenylcarbamate (50 mg, 0.14 mmol) and 7,8-dimethoxy-1-phenyl-2,3,4,5-tetrahydro-3H-benzazepine (39.6 mg, 0.14 mmol) were mixed in anhydrous THF (0.7 ml), and then DBU (30 μl, 0.20 mmol) was added dropwise to this reaction solution. After stirring for 1 hour at room temperature, the product was extracted with methylene chloride (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:3, v/v) to give 21.6 mg (Yield 28.4%) of the title compound as a solid.

m.p.: 108-110° C.

¹H NMR (500 MHz, CDCl₃): δ ppm 6.48-7.85 (m, 15H), 4.60 (m, 1H), 4.30 (m, 1H), 3.87 (m, 5H), 3.80 (m, 1H), 3.69 (s, 3H), 3.30 (m, 1H), 2.88 (m, 1H)

### EXAMPLE 9

Synthesis of 3-[N-(diphenylmethyl)aminocarbonyl]-7,8-dimethoxy-1-phenyl-2,3,4,5-tetrahydro-3H-benzazepine

N-(diphenylmethyl)phenylcarbamate (94.2 mg, 0.31 mmol) and 7,8-dimethoxy-1-phenyl-2,3,4,5-tetrahydro-3H-benzazepine (80 mg, 0.28 mmol) were mixed in anhydrous THF (5 ml), and then DBU (45.6 μl, 0.30 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 106 mg (Yield 76.3%) of the title compound as a solid.

m.p.: 155-158° C.

¹H NMR (300 MHz, CDCl₃): δ ppm 7.01-7.30 (m, 15H), 6.66 (s, 1H), 6.40 (s, 1H), 6.01 (d, 1H, J=6.86 Hz), 4.74 (d, 1H, J=6.86 Hz), 4.51 (dd, 1H, J=8.61 Hz, J=5.31 Hz), 3.89 (s, 3H), 3.69 (s, 3H), 3.53-3.84 (m, 3H), 3.22 (m, 1H), 2.84 (m, 1H)

### EXAMPLE 10

Synthesis of 3-[N-(diphenylmethyl)aminocarbonyl]-7,8-dimethoxy-1-(4-fluoro)phenyl-2,3,4,5-tetrahydro-3H-benzazepine

N-(diphenylmethyl)phenylcarbamate (51.7 mg, 0.17 mmol) and 7,8-dimethoxy-1-(4-fluoro)phenyl-2,3,4,5-tetrahydro-3H-benzazepine (51.4 mg, 0.17 mmol) were mixed in anhydrous THF (4 ml), and then DBU (25.5 μl, 0.17 mmol) was added dropwise to this reaction solution. After stirring for 18 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 54.4 mg (Yield 62.5%) of the title compound as a solid.

m.p.: 187-188° C.

¹H NMR (300 MHz, CDCl₃): δ ppm 6.88-7.30 (m, 14H), 6.66 (s, 1H), 6.44 (s, 1H), 6.02 (d, 1H, J=6.85 Hz), 4.72 (d, 1H, J=6.85 Hz), 4.48 (t, 1H), 4.07 (m, 1H), 3.89 (s, 3H), 3.71 (s, 3H), 3.61-3.85 (m, 2H), 3.55 (m, 1H), 3.20 (m, 1H), 2.86 (m, 1H)

### EXAMPLE 11

Synthesis of 3-[N-(diphenylmethyl)aminocarbonyl]-7,8-dimethoxy-1-(4-chloro)phenyl-2,3,4,5-tetrahydro-3H-benzazepine

N-(diphenylmethyl)phenylcarbamate (56.4 mg, 0.19 mmol) and 7,8-dimethoxy-1-(4-chloro)phenyl-2,3,4,5-tetrahydro-3H-benzazepine (59.1 mg, 0.19 mmol) were mixed in anhydrous THF (3 ml), and then DBU (27.8 μl, 0.19 mmol) was added dropwise to this reaction solution. After stirring for 16 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 75.9 mg (Yield 77.5%) of the title compound as a solid.

m.p.: 205-207° C.

¹H NMR (300 MHz, CDCl₃): δ ppm 6.99-7.30 (m, 14H), 6.66 (s, 1H), 6.44 (s, 1H), 6.02 (d, 1H, J=6.86 Hz), 4.74 (d, 1H, J=6.86 Hz), 4.48 (dd, 1H, J=8.45 Hz, J=5.15 Hz), 4.06 (dd, 1H, J=14.85 Hz, J-5.15 Hz), 3.79-3.89 (m, 4H), 3.71 (s, 3H), 3.53-3.61 (m, 2H), 3.18 (m, 1H), 2.87 (m, 1H)

### EXAMPLE 12

Synthesis of 1-[N-(5,6-dimethyl-2-methoxypyridin-3-yl)aminocarbonyl]-4-diphenylmethylene-piperidine

N-(5,6-dimethyl-2-methoxypyridin-3-yl)phenylcarbamate (50 mg, 0.184 mmol) and 4-diphenylmethylene-piperidine. HCl (52.5 mg, 0.184 mmol) were mixed in anhydrous THF (5 ml), and then DBU (60.7 μl, 0.41 mmol) was added dropwise to this reaction solution. After stirring for 2 hours at room temperature, the product was extracted with ethyl acetate (50 ml×3). The organic solution was washed with aqueous sodium chloride solution and water, dried over anhydrous magnesium sulfate, and concentrated under reduced pressure. The residue was crystallized from ethyl acetate and hexane to give 44.7 mg (Yield 51.3%) of the title compound as a solid.

m.p.: 190-192° C.

¹H NMR (300 MHz, CDCl₃): δ ppm 8.16 (s, 1H), 7.11-7.32 (m, 10H), 6.86 (br, 1H), 3.93 (s, 3H), 3.55 (t, 4H, J=5.67 Hz), 2.46 (t, 4H, J=5.67 Hz), 2.32 (s, 3H), 2.17 (s, 3H)

### EXAMPLE 13

Synthesis of 1-[N-(diphenylmethyl)aminocarbonyl]-4-diphenylmethylene-piperidine

N-(diphenylmethyl)phenylcarbamate (100 mg, 0.33 mmol) and 4-diphenylmethylene-piperidine. HCl (94.2 mg, 0.33 mmol) were mixed in anhydrous THF (5 ml), and then DBU (108 μl, 0.72 mmol) was added dropwise to this reaction solution. After stirring for 3 hours at room temperature, the product was extracted with methylene chloride (50 ml×3). The organic solution was washed with aqueous sodium chloride solution and water, dried over anhydrous magnesium sulfate, and concentrated under reduced pressure. The residue was crystallized from ethyl acetate and diethylether to give 100.7 mg (Yield 66.5%) of the title compound as a solid.

m p.: 206° C.

¹H NMR (300 MHz, CDCl₃): δ ppm 7.09-7.34 (m, 20H), 6.16 (d, 1H, J=6.78 Hz), 4.99 (d, 1H, J=6.78 Hz), 3.48 (t, 4H, J=5.67 Hz), 2.40 (t, 4H, J=5.67 Hz)

¹³C NMR (75 MHz, CDCl₃): δ ppm 184.45, 151.07, 146.37, 142.65, 129.60, 128.59, 128.09, 127.35, 127.22, 126.58, 58.37, 45.27, 31.29

### EXAMPLE 14

Synthesis of 1-[N-(2,2-diphenylethan-1-yl)aminocarbonyl]-4-diphenylmethylene-piperidine

N-(2,2-diphenylethan-1-yl)phenylcarbamate (500 mg, 1.57 mmol) and 4-diphenyl methylene-piperidine. HCl (450 mg, 1.57 mmol) were mixed in anhydrous THF (5 ml), and then DBU (0.5 ml, 3.31 mmol) was added dropwise to this reaction solution. After stirring for 3 hours at room temperature, the product was extracted with methylene chloride (50 ml×3). The organic solution was washed with aqueous sodium chloride solution and water, dried over anhydrous magnesium sulfate, and concentrated under reduced pressure. The residue was crystallized from ethyl acetate and diethylether to give 519.4 mg (Yield 70.0%) of the title compound as a solid.

m.p.: 158-160° C.

¹H NMR (300 MHz, CDCl₃): δ ppm 7.06-7.32 (m, 20H), 4.42 (t, 1H, J=5.69 Hz), 4.23 (t, 1H, J=7.86 Hz), 3.86 (dd, 2H, J=5.69 Hz, J=7.86 Hz), 3.25 (dd, 4H, J=5.70 Hz, J=5.85 Hz), 2.27 (dd, 4H, J=5.67, J=5.85 Hz)

¹³C NMR (75 MHz, CDCl₃): δ ppm 157.22, 142.12, 141,93, 137.37, 133.69, 129.54, 128.58, 128.07, 128.01, 126.63, 126.49, 50.85, 45.22, 44.91, 31.08

### EXAMPLE 15

Synthesis of 1-[N-(5,6-dimethyl-2-methoxypyridin-3-yl)aminocarbonyl]-4,4-diphenyl-piperidine

N-(5,6-dimethyl-2-methoxypyridin-3-yl)phenylcarbamate (60 mg, 0.25 mmol) and 4,4-diphenylpiperidine (60 mg, 0.25 mmol) were mixed in anhydrous THF (10 ml), and then DBU (48 μl, 0.32 mmol) was added dropwise to this reaction solution. After stirring for 2 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 56 mg (Yield 54%) of the title compound as a syrup.

¹H NMR (300 MHz, CDCl₃): δ ppm 8.11 (s, 1H), 7.17, 6.84 (2m, 10H), 6.40 (br, 1H), 3.96 (s, 3H), 4.24, 3.57, 2.72, 2.48, 1.90, 1.74 (m, 8H), 2.33, 2.16 (s, 6H)

### EXAMPLE 16

Synthesis of 1-[N-(2-methoxyquinoxalin-3-yl)aminocarbonyl]-4,4-diphenyl-piperidine

N-(2-methoxyquinoxalin-3-yl)phenylcarbamate (73.8 mg, 0.25 mmol) and 4,4-diphenylpiperidine (60 mg, 0.25 mmol) were mixed in anhydrous THF (10 ml), and then DBU (48 μl, 0.32 mmol) was added dropwise to this reaction solution. After stirring for 2 hours at room temperature, the product was extracted with ethyl acetate (50 ml×3). The organic solution was washed with aqueous sodium chloride solution and water, dried over anhydrous magnesium sulfate, and concentrated under reduced pressure. The residue was crystallized from ethyl acetate and hexane to give 72 mg (Yield 65.7%) of the title compound as a solid.

m.p.: 202-204° C.

¹H NMR (300 MHz, CDCl₃): δ ppm 14.37 (br, 1H), 7.79-7.19 (m, 14H), 4.14 (s, 3H), 4.21, 3.68, 2.57, 1.93, 1.62 (5m, 8H)

### EXAMPLE 17

Synthesis of 1-[N-(diphenylmethyl)aminocarbonyl]-4,4-diphenyl-piperidine

N-(diphenylmethyl)phenylcarbamate (96 mg, 0.32 mmol) and 4,4-diphenylpiperidine (76 mg, 0.32 mmol) were mixed in anhydrous THF (10 ml), and then DBU (64 μl, 0.43 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was crystallized from ethyl acetate to give 94 mg (Yield 65.8%) of the title compound as a solid.

m.p.: 232-234° C.

¹H NMR (300 MHz, CDCl₃): δ ppm 7.24 (m, 10H), 6.12 (d, 1H), 4.95 (d, 1H, J=6.42 Hz), 3.47, 2.42 (2m, 8H)

### EXAMPLE 18

Synthesis of 1-[N-(diphenylmethyl)aminocarbonyl]-4-hydroxycarbonyl-4-phenyl-piperidine

N-(diphenylmethyl)phenylcarbamate (100 mg, 0.33 mmol) and 4-hydroxycarbonyl-4-phenyl-piperidine (124.5 mg, 0.33 mmol) were mixed in anhydrous THF (10 ml), and then DBU (49 μl, 0.33 mmol) was added dropwise to this reaction solution. After stirring for 3 hours at room temperature, the product was extracted with methylene chloride (50 ml) and water of pH 4 (50 ml). The aqueous layer was adjusted to pH 7 and crystallized to give 81.5 mg (Yield 59.6%) of the title compound as a solid.

m.p. 208° C.

¹H NMR (300 MHz, CDCl₃): δ ppm 7.22-7.42 (m, 15H), 6.09 (d, 1H, J=8.61 Hz), 3.98 (d, 2H, J=13.56 Hz), 2.98 (dd, 2H, J=11.70, J=12.27 Hz), 2.40 (d, 2H, J=12.81 Hz), 1.73 (dd, 2H, J=10.44 Hz, J=10.62 Hz)

¹³C NMR (75 MHz, CDCl₃): δ ppm 175.24, 156.95, 143.44, 142.91, 128.53, 128.15, 127.52, 126.93, 126.64, 125.74, 57.48, 48.83, 41.85, 33.35

### EXAMPLE 19

Synthesis of 1-[N-(5-ethyl-6-methyl-2-methoxypyridin-3-yl)aminocarbonyl]-4-hydroxycarbonyl-4-phenyl-piperidine

N-(5-ethyl-6-methyl-2-methoxypyridin-3-yl)phenylcarbamate (60 mg, 0.21 mmol) and 4-hydroxycarbonyl-4-phenyl-piperidine (79.1 mg, 0.21 mmol) were mixed in anhydrous THF (6 ml), and then DBU (65.8 μl, 0.44 mmol) was added dropwise to this reaction solution. After stirring for 3 hours at room temperature, the product was extracted with methylene chloride (50 ml) and water of pH 4 (50 ml). The aqueous layer was adjusted to pH 7 and crystallized to give 79.3 mg (Yield 95.2%) of the title compound as a solid.

m.p.: 173-174° C.

¹H NMR (300 MHz, CDCl₃): δ ppm 7.71 (s, 1H), 7.23-7.67 (m, 5H), 3.83 (s, 3H), 3.74 (d, 2H, J=13.53 Hz), 3.04 (dd, 2H, J=12.09 Hz, J=12.27 Hz), 2.48 (q, 2H, J=7.5 Hz), 2.42 (d, 2H, J=13.02 Hz), 2.31 (s, 3H), 1.78 (dd, 2H, J=10.44 Hz, J=10.62 Hz), 1.08 (t, 3H, J=7.50 Hz)

¹³C NMR (75 MHz, CDCl₃): δ ppm 175.16, 155.09, 152.85, 145.89, 142.78, 131.24, 128.93, 128.55, 126.97, 125.75, 120.97, 53.01, 48.76, 41.88, 33.25, 24.33, 20.80, 14.49

### EXAMPLE 20

Synthesis of 1-[N-(2,2-diphenylethan-1-yl)aminocarbonyl]-4-hydroxy carbonyl-4-phenyl-piperidine

N-(2,2-diphenylethan-1-yl)phenylcarbamate (l g, 3.15 mmol) and 4-hydroxy carbonyl-4-phenyl-piperidine (1.19 g, 3.15 mmol) were mixed in anhydrous THF (10 ml), and then DBU (1 ml, 6.62 mmol) was added dropwise to this reaction solution. After stirring for 3 hours at room temperature, the product was extracted with methylene chloride (100 ml) and water of pH 4 (100 ml). The aqueous layer was adjusted to pH 7 and crystallized to give 0.75 g (Yield 55.8%) of the title compound as a solid.

m.p. 173-174° C.

¹H NMR (300 MHz, CDCl₃): δ ppm 7.01-7.31 (m, 15H), 6.50 (t, 1H, J=5.49 Hz), 4.22 (t, 1H, J=7.71 Hz), 3.65 (d, 2H, J=13.74 Hz), 3.58 (dd, 2XL J=5.49 Hz, J=7.71 Hz), 2.72 (dd, 2H, J=11.70 Hz, J=11.9 Hz), 2.14 (d, 2H, J=13.2 Hz), 1.39 (dd, 2H, J=9.9 Hz, J=11.16 Hz)

¹³C NMR (75 MHz, CDCl₃): δ ppm 175.21, 157.33, 143.11, 142.93, 128.44, 128.26, 128.00, 126.87, 126.12, 125.67, 50.45, 48.84, 44.69, 41.86, 32.91

### EXAMPLE 21

Synthesis of 1-[N-(5-ethyl-6-methyl-2-methoxypyridin-3-yl)aminocarbonyl]spiro[isobenzofuran-1(3H),4-piperidin]-3-one

N-(5-ethyl-6-methyl-2-methoxypyridin-3-yl)phenylcarbamate (40 mg, 0.20 mmol) and spiro[isobenzofuran-1(3H),4-piperidin]-3-one (56.3 mg, 0.20 mmol) were mixed in anhydrous THF (10 ml), and then DBU (35 μl, 0.22 mmol) was added dropwise to this reaction solution. After stirring for 6 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:5, v/v) to give 64 mg (Yield 88.2%) of the title compound as a solid.

¹H NMR (300 MHz, CDCl₃): δ ppm 8.20 (s, 1H), 7.91 (d, 1H), 7.71, 7.57 (t, 2H), 7.27 (d, 1H), 4.19 (m, 2H), 3.97 (s, 3H), 3.45 (m, 2H), 2.57 (m, 2H), 2.25 (s, 3H), 2.16 (m, 2H), 1.80 (m, 2H), 1.19 (t, 3H)

### EXAMPLE 22

Synthesis of 1-[N-(2-methoxyquinoxalin-3-yl)aminocarbonyl]spiro[isobenzofuran-1(3H),4-piperidin]-3-one

N-(2-methoxyquinoxalin-3-yl)phenylcarbamate (26 mg, 0.13 mmol) and spiro [isobenzofuran-1(3H),4-piperidin]-3-one (37 mg, 0.13 mmol) were mixed in anhydrous THF (5 ml), and then DBU (24 μl, 0.16 mmol) was added dropwise to this reaction solution. After stirring for 5 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 43 mg (Yield 89.2%) of the title compound as a solid.

¹H NMR (300 MHz, CDCl₃): δ ppm 7.9-7.21 (m, 8H), 5.04 (m, 2H), 4.25 (m, 2H), 4.16 (m, 2H), 4.12 (s, 3H), 3.56 (m, 2H), 2.42 (m, 2H), 2.25 (s, 3H), 2.16 (m, 2H), 1.80 (m, 2H), 1.19 (t, 3H)

### EXAMPLE 23

Synthesis of 1-[(5,6-dimethyl-2-methoxypyridin-3-yl)aminocarbonyl]-4-(diphenylmethyl)piperazine

N-(5,6-dimethyl-2-methoxypyridin-3-yl)phenylcarbamate (54.4 mg, 0.2 mmol) and 1-(diphenylmethyl)piperazine (52 mg, 0.2 mmol) were mixed in anhydrous THF (10 ml), and then DBU (40 μl, 0.27 mmol) was added dropwise to this reaction solution. After stirring for 2 hours at room temperature, the product was extracted with ethyl acetate (50 ml ×3). The organic solution was washed with aqueous sodium chloride solution and water, dried over anhydrous magnesium sulfate, and concentrated under reduced pressure. The residue was crystallized from ethyl acetate and hexane to give 71 mg (Yield 88.3%) of the title compound as a solid.

m.p.: 132-134° C.

¹H NMR (300 MHz, CDCl₃): δ ppm 8.14 (s, 1H), 7.43, 7.29, 7.19 (3m, 10H), 4.25 (s, 1H), 3.91 (s, 3H), 3.49 (m, 4H), 2.44 (m, 4H), 2.16 (s, 3H), 2.04 (s, 3H)

### EXAMPLE 24

Synthesis of 1-[(5,6-dimethyl-2-methoxypyridin-3-yl)amino-N-cyanocarboimidate]-4-(3,5-dimethylphenyl)piperazine

1-(3,5-Dimethylphenyl)piperazine (400 mg, 2.1 mmol) and diphenyl-N-cyano carboimidate (550.9 mg, 2.3 mmol) were mixed in anhydrous DMF (10 ml), and then 60% NaH (92.5 mg, 2.3 mmol) was added dropwise to this reaction solution. After stirring for 30 minutes at room temperature, the product was extracted with ethyl acetate (100 ml×3). The organic solution was washed with aqueous sodium chloride solution and water, dried over anhydrous magnesium sulfate, and concentrated under reduced pressure. The residue was crystallized from ethyl acetate and diethylether to give 523 mg (Yield 67.9%) of the intermediate compound 1-(phenoxy-N-cyanocarboimidate)-4-(3,5-dimethylphenyl) piperazine as a solid. The intermediate (117.8 mg, 0.32 mmol) thus obtained and 5,6-dimethyl-2-methoxy-3-amino-pyridine (48.9 mg, 0.32 mmol) were mixed in anhydrous THF (10 ml), and then 60% NaH (34 mg, 0.85 mmol) was added dropwise to this reaction solution. After stirring for 24 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 62.8 mg (Yield 50%) of the title compound as a solid.

m.p.: 214-217° C.

¹H NMR (300 MHz, CDCl₃): δ ppm 7.19 (s, 1H), 6.73 (s, 1H), 6.57 (s, 1H), 6.52 (s, 2H), 4.20 (s, 3H), 3.58 (t, 4H, J=5.13 Hz), 3.14 (t, 4H, J=5.13 Hz), 2.36 (s, 3H), 2.27 (s, 6H), 2.18 (s, 3H)

### EXAMPLE 25

Synthesis of 1-[(5,6-dimethyl-2-methoxypyridin-3-yl)aminocarbonyl]-4-[4,6-bis(propylamino)-1,3,5-triazin-2-yl]piperazine

N-(5,6-dimethyl-2-methoxypyridin-3-yl)phenylcarbamate (60 mg, 0.22 mmol) and 1-[4,6-bis(propylamino)-1,3,5-triazin-2-yl]piperazine (74.3 mg, 0.27 mmol) were mixed in anhydrous THF (10 ml), and then DBU (40.4 μl, 0.27 mmol) was added dropwise to this reaction solution. After stirring for 30 minutes at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 102 mg (Yield 98%) of the title compound as a solid.

m.p.: 140-143° C.

¹H NMR (300 MHz, CDCl₃): δ ppm 8.13 (s, 1H), 6.84 (s, 1H), 4.25 (s, 2H), 3.97 (s, 3H), 3.72 (s, 4H), 3.55 (s, 4H), 3.37 (s, 2H), 2.33 (s, 3H), 2.18 (s, 3H), 1.63 (m, 2H), 1.46 (m, 2H), 0.97 (t, 3H), 0.93 (t, 3H)

### EXAMPLE 26

Synthesis of 1-[(2-methoxy-5-cyanophenyl)aminocarbonyl]-4-(3,5-dimethyl phenyl)piperazine

N-(2-methoxy-5-cyanophenyl)phenylcarbamate (500 mg, 1.73 mmol) and 1-(3,5-dimethylphenyl)piperazine (330 mg, 1.73 mmol) were mixed in anhydrous THF (10 ml), and then DBU (0.26 ml, 1.73 mmol) was added dropwise to this reaction solution. After stirring for 24 hours at room temperature, the product was extracted with ethyl acetate (100 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 483 mg (Yield 98%) of the title compound as a solid.

¹H NMR (300 MHz, CDCl₃): δ ppm 9.09 (s, 1H), 7.89 (d, 1H, J=8.97 Hz), 7.17 (s, 1H), 6.89 (d, 1H, J=8.97 Hz), 6.57 (s, 3H), 4.00 (s, 3H), 3.67 (d, 4H, J=3.66 Hz), 3.23 (d, 4H, J=3.84 Hz), 2.29 (s, 6H)

### EXAMPLE 27

Synthesis of 1-[(1,4-benzodioxan-6-yl)amino-N-cyanocarboimidate]-4-(3,5-dimethylphenyl)piperazine

(1,4-Benzodioxan-6-yl)amine (450 mg, 3 mmol) and diphenyl-N-cyano carboimidate (857 mg, 3.2 mmol) were mixed in anhydrous DMF (10 ml), the resulting solution was cooled to 0° C., and then NaH (86.4 mg, 3.2 mmol) was added dropwise to this reaction solution. After stirring for 4 hours at room temperature, the product was extracted with ethyl acetate (100 ml×3). The organic solution was washed with aqueous sodium chloride solution and water, dried over anhydrous magnesium sulfate, and concentrated under reduced pressure. Without purification, the intermediate compound, N-(1,4-benzodioxan-6-yl)aminophenyl-N-cyanocarboimidate (295 mg, 1.00 mmol) and 1-(3,5-dimethylphenyl)piperazine (209 mg, 1.2 mmol) were mixed in anhydrous DMF (3 ml) and then NaH (26 mg, 0.85 mmol) was added dropwise to this reaction solution. After stirring for 4 hours at room temperature, the product was extracted with ethyl acetate (50 ml×3). The organic solution was washed with aqueous sodium chloride solution and water, dried over anhydrous magnesium sulfate, and concentrated under reduced pressure. The residue was crystallized from petrolium ether and methylene chloride to give 157 mg (Yield 40%) of the title compound as a solid.

m.p.: 201-203° C.

¹H NMR (300 MHz, CDCl₃): δ ppm 10.66 (s, 1), 6.62 (s, 2H), 6.54 (s, 1H), 6.50 (s, 2H), 4.24 (s, 4H), 3.51 (t, 4H, J=5.03 Hz), 3.09 (t, 4H, J=5.03 Hz), 2.26 (s, 6H)

### EXAMPLE 28

Synthesis of 1-[(benzo-1,2,4-triazin-1-N-oxide-3-yl)aminocarbonyl]-4-(3,5-dimethylphenyl)piperazine

3-Amino-benzo-1,2,4-triazin-1-N-oxide (45 mg, 0.3 mmol), 1-(3,5-dimethylphenyl) piperazine (57 mg, 0.3 mmol), and 1,1′-carbonyldiimidazole (81 mg, 0.5 mmol) were mixed in anhydrous THF (20 ml), and then DBU (112 μl, 0.7 mmol) was added dropwise to this reaction solution. After stirring for 12 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 75 mg (Yield 68.7%) of the title compound as a solid.

¹H NMR (300 MHz, CDCl₃): δ ppm 7.90, 7.23, 7.11, 6.56 (4m, 7H, Ph), 3.75 (m, 4H), 3.22 (m, 4H), 2.27 (s, 6H)

### EXAMPLE 29

Synthesis of 1-[(benzo-1,2,4-triazin-1-N-oxide-3-yl)aminocarbonyl]-4-diphenyl methyl)piperazine

3-Amino-benzo-1,2,4-triazin-1-N-oxide (30 mg, 0.2 mmol), 1-(diphenylmethyl) piperazine (52 mg, 0.2 mmol), and 1,1′-carbonyldiimidazole (49 mg, 0.32 mmol) were mixed in anhydrous THF (10 ml), and then DBU (48 μl, 0.32 mmol) was added dropwise to this reaction solution. After stirring for 4 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 43 mg (Yield 50.4%) of the title compound as a syrup.

¹H NMR (300 MHz, CDCl₃): δ ppm 7.83-7.05 (m, 14H), 4.28 (s, 1H), 3.61, 2.46 (2m, 8H)

### EXAMPLE 30

Synthesis of 1-[(2-phenylvinyl)aminocarbonyl]-4-(3,5-dimethylphenyl) piperazine

(2-Phenylvinyl)amine (59.5 mg, 0.5 mmol), 1-(3,5-dimethylphenyl)piperazine (105 mg, 0.5 mmol), and 1,1′-carbonyldiimidazole (81 mg, 0.5 mmol) were mixed in anhydrous TBT (20 ml), and then DBU (288 μl, 1.8 mmol) was added dropwise to this reaction solution. After stirring for 6 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 110.8 mg (Yield 66.5%) of the title compound as a syrup.

¹H NMR (300 MHz, CDCl₃): δ ppm 7.70 (d, J=15.29 Hz, 1H), 7.24-7.55 (m, 5H), 6.91 (d, J=15.39 Hz, 1H), 6.57 (m, 3H), 3.81, 3.19 (t, J=5.31 Hz, 8H), 2.28 (s, 6H)

### EXAMPLE 31

Synthesis of 1-[(diphenylmethyl)aminocarbonyl]-4-(diphenylmethyl) piperazine

N-(diphenylmethyl)phenylcarbamate (121.2 mg, 0.4 mmol) and 1-(diphenylmethyl) piperazine (104 mg, 0.4 mmol) were mixed in anhydrous THF (10 ml), and then DBU (80 μl, 0.53 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was washed with methylene chloride to give 136 mg (Yield 73.6%) of the title compound as a solid.

m.p.: 216-218° C.

¹H NMR (300 MHz, CDCl₃): δ ppm 7.40, 7.23 (2m, 20H), 6.02 (d, 1H, J=8.4 Hz), 4.30 (s, 1H), 3.37 (m, 4H), 2.24 (m, 4H)

### EXAMPLE 32

Synthesis of 1-[(diphenylmethyl)aminocarbonyl]-4-(3,5-dimethylphenyl) piperazine

N-(diphenylmethyl)phenylcarbamate (50 mg, 0.16 mmol) and 1-(3,5-dimethyl phenyl)piperazine (31.4 mg, 0.16 mmol) were mixed in anhydrous THF (10 ml) and then DBU (54 μl, 0.36 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was crystallized from ethyl acetate to give 37.4 mg (Yield 56.8%) of the title compound as a solid.

m.p.: 173-174° C.

¹H NMR (300 MHz, CDCl₃): δ ppm 7.23-7.35 (m, 10H), 6.54 (s, 3H), 6.16 (d, 1H, J=6.78 Hz), 5.05 (d, 1H, J=6.78 Hz), 3.55 (t, 4H, J=5.31 Hz), 3.15 (t, 4H, J=5.31 Hz), 2.27 (s, 6H)

### EXAMPLE 33

Synthesis of 1-[(diphenylmethyl)aminothiocarbonyl]-4-(diphenylmethyl) piperazine

Aminodiphenylmethane (500 mg, 2.7 mmol), 1-(diphenylmethyl)piperazine (486 mg, 2.7 mmol), and 1,1′-thiocarbonyldiimidazole (486 mg, 2.7 mmol) were mixed in anhydrous THF (10 ml), and then DBU (820 μl, 5.5 mmol) was added dropwise to this reaction solution. After stirring for 19 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:20, v/v) to give 144 mg (Yield 11.5%) of the title compound.

m.p.: 118-120° C.

¹H NMR (300 MHz, CDCl₃): δ ppm 7.15-7.41 (m, 20H), 6.94 (d, 1H, J=7.41 Hz), 5.90 (d, 1H, J=7.41 Hz), 4.24 (s, 1H), 3.82 (t, 4H, J=5.1 Hz), 2.45 (t, 413, J=5.1 Hz)

### EXAMPLE 34

Synthesis of 1-[(diphenylmethyl)aminocarbonyl]-4-[4,6-bis(propylamino)-1,3,5-triazin-2-yl]piperazine

N-(diphenylmethyl)phenylcarbamate (29.6 mg, 0.098 mmol) and 1-[4,6-bis(propylamino)-1,3,5-triazin-2-yl]piperazine (10.5 mg, 0.098 mmol) were mixed in anhydrous THF (1 ml), and then DBU (14.6 μl, 0.098 mmol) was added dropwise to this reaction solution. After stirring for 4 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 9.6 mg (Yield 52.3%) of the title compound as a syrup.

¹H NMR (300 MHz, CDCl₃): δ ppm 7.19-7.35 (m, 10H), 6.16 (d, 1H, J=6.69 Hz), 5.01 (d, 1H, J=6.69 Hz), 4.24 (s, 2H), 3.84 (s, 4H), 3.45 (s, 4H), 3.34 (s, 2H), 1.58 (m, 2H), 1.44 (m, 2H), 0.94 (t, 3H, J=7.24 Hz), 0.94 (t, 3H, J=7.23 Hz)

### EXAMPLE 35

Synthesis of 1-[(diphenylmethyl)amino-N-cyanocarboimidate]-4-(3,5-dimethylphenyl)piperazine

Aminodiphenylmethane (0.56 ml, 3.27 mmol) and diphenyl-N-cyanocarboimidate (936 mg, 3.93 mmol) were mixed in anhydrous DMF (12 ml), and then TEA (0.55 ml, 3.93 mmol) was added dropwise to this reaction solution. After stirring for 4 hours at room temperature, the product was extracted with ethyl acetate (100 ml×3). The organic solution was washed with aqueous sodium chloride solution and water, dried over anhydrous magnesium sulfate, and concentrated under reduced pressure. The residue was crystallized from ethyl acetate and diethylether to give 1.045 g (Yield 97.6%) of the intermediate compound N-(diphenylmethyl)amino-phenoxy-N-cyanocarboimidate as a solid. The intermediate (200 mg, 0.61 mmol) thus obtained and 1-(3,5-dimethylphenyl)piperazine (116.2 mg, 0.61 mmol) were mixed in anhydrous DMF (3 ml), and then 60% NaH (24.4 mg, 0.61 mmol) was added dropwise to this reaction solution. After stirring for 1 hour at 80° C., the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 152.1 mg (Yield 58.6%) of the title compound.

m.p.: 205-208° C.

¹H NMR (300 MHz, CDCl₃): δ ppm 7.23-7.34 (m, 10H), 6.56 (s, 1H), 6.50 (s, 2H), 6.24 (d, 1H, J=8.03 Hz), 5.96 (d, 1H, J=8.03 Hz), 3.62 (t, 4H), 3.14 (t, 4H,), 2.26 (s, 6H)

¹³C NMR (75 MHz, CDCl₃): δ ppm 160.10, 150.65, 140.79, 138.84, 128.82, 127.85, 127.43, 122.56, 117.29, 114.49, 61.11, 49.21, 46.96, 21.61

### EXAMPLE 36

Synthesis of 1-[(2,2-diphenylethan-1-yl)aminocarbonyl]-4-(3,5-dimethyl phenyl)piperazine

N-(2,2-diphenylethan-1-yl)phenylcarbamate (50 mg, 0.16 mmol) and 1-(3,5-dimethylphenyl)piperazine (30.0 mg, 0.16 mmol) were mixed in anhydrous THF (5 ml) and then DBU (26 μl, 0.17 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was crystallized from hexane to give 43.2 mg (Yield 66.2%) of the title compound as a solid.

m.p.: 174-175° C.

¹H NMR (300 MHz, CDCl₃): δ ppm 7.19-7.33 (m, 10H), 6.51 (s, 3H), 4.41 (br, 1H), 4.26 (t, 1H, J=7.7 Hz), 3.88 (dd, 2H, J=5.7, 7.7 Hz), 3.35 (t, 4H, J=4.77 Hz), 3.05 (t, 4H, J=4.77 Hz), 2.26 (s, 6H)

¹³C NMR (75 MHz, CDCl₃): δ ppm 157.38, 151.08, 142.10, 138.72, 128.66, 128.12, 126.73, 122.21, 114.42, 50.86, 49.19, 45.22, 43.67, 21.57

### EXAMPLE 37

Synthesis of 1-[(2,2-diphenylethan-1-yl)aminocarbonyl]-4-(diphenylmethyl) piperazine

N-(2,2-diphenylethan-1-yl)phenylcarbamate (50 mg, 0.16 mmol) and 1-(diphenylmethyl)piperazine (39.8 mg, 0.16 mmol) were mixed in anhydrous THF (5mi), and then DBU (26 μl, 0.17 mmol) was added dropwise to this reaction solution. After stirring for 2 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=3:7, v/v) to give 63.1 mg (Yield 89.2%) of the title compound as a solid.

m.p.: 175-176° C.

¹H NMR (300 MHz, CDCl₃): δ ppm 7.13-7.38 (m, 20H), 4.34 (br, 1H), 4.20 (t, 1H, J=7.7 Hz), 4.16 (s, 1H), 3.83 (dd, 2H, J=5.9, 7.7 Hz), 3.18 (t, 4H, J=4.92 Hz), 2.27 (t, 4H, J=4.92 Hz)

¹³C NMR (75 MHz, CDCl₃): δ ppm 157.52, 142.33, 142.18, 128.67, 128.59, 128.15, 127.85, 127.10, 126.72, 76.01, 51.46, 50.89, 45.18, 43.82

### EXAMPLE 38

Synthesis of 1-[(diphenylmethyl)aminocarbonyl]-4-(4,6-bis-allylamino-[1,3,5]triazin-2-yl)piperazine

N-(diphenylmethyl)phenylcarbamate (70 mg, 0.23 mmol) and 1-(4,6-bis-allylamino-[1,3,5]triazin-2-yl)piperazine (63.5 mg, 0.23 mmol) were mixed in anhydrous THF (4 ml), and then DBU (34.5 μl, 0.23 mmol) was added dropwise to this reaction solution. After stirring for 2 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 77.4 mg (Yield 69.4%) of the title compound as a solid.

m.p.: 182-184° C.

¹H NMR (300 MHz, CDCl₃): δ ppm 7.25-7.32 (m, 10H), 6.16 (d, 1H, J=4.58 Hz), 5.89 (m, 2H), 5.19 (d, 2H), 5.09 (d, 2H), 5.04 (d, 1H, J=4.58 Hz), 3.99 (s, 4H), 3.78 (s, 4H), 3.43 (s, 4H)

¹³C NMR (75 MHz, CDCl₃): δ ppm 165.06, 156.70, 142.40, 135.19, 128.59, 127.34, 127.27, 115.62, 58.36, 43.62, 43.15, 42.62, 29.65

EI MS, m/e=484

### EXAMPLE 39

Synthesis of 1-[(diphenylmethyl)aminocarbonyl]-4-[4,6-bis-(allylcyclohexyl-amino)-[1,3,5]triazin-2-yl]piperazine

N-(diphenylmethyl)phenylcarbamate (35.1 mg, 0.12 mmol) and 1-[4,6-bis-(allylcyclohexyl-amino)-[1,3,5]triazin-2-yl]piperazine (50.8 mg, 0.12 mmol) were mixed in anhydrous THF (4 ml), and then DBU (17.3 μl, 0.12 mmol) was added dropwise to this reaction solution. After stirring for 15 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 61.0 mg (Yield 81.3%) of the title compound as a solid.

m.p.: 200-202° C.

¹H NMR (300 MHz, CDCl₃): δ ppm 7.25-7.35 (m, 10H), 6.17 (d, 1H, J=6.6 Hz), 5.83-5.92 (m, 2H), 5.11 (d, 2H), 5.00 (d, 2H), 4.96 (d, 1H, J=6.6 Hz), 4.43 (br, 2H), 4.21 (d, 4H), 3.77 (s, 4H), 3.44 (s, 4H), 1.12-1.77 (m, 20H)

### EXAMPLE 40

Synthesis of 1-[(diphenylmethyl)aminocarbonyl]-4-{4,6-bis-[ethyl-(2-methylallyl)amino]-[1,3,5]triazin-2-yl}piperazine

N-(diphenylmethyl)phenylcarbamate (50.9 mg, 0.17 mmol) and 1-{4,6-bis-[ethyl-(2-methylallyl)amino]-[1,3,5]triazin-2-yl}piperazine (60.3 mg, 0.17 mmol) were mixed in anhydrous THF (4 ml), and then DBU (25.1 μl, 0.17 mmol) was added dropwise to this reaction solution. After stirring for 15.5 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 86.5 mg (Yield 90.7%) of the title compound as a solid.

m.p.: 178-180° C.

¹H NMR (300 MHz, CDCl₃): δ ppm 7.19-7.35 (m, 10H), 6.16 (d, 1H, J=6.48 Hz), 5.00 (d, 1H, J=6.48 Hz), 4.79 (d, 4H), 4.12 (s, 4H), 3.79 (s, 4H), 3.45 (s, 8H), 1.67 (s, 6H), 1.11 (s, 6H)

### EXAMPLE 41

Synthesis of 1-[(diphenylmethyl)aminocarbonyl]-4-(4,6-bis-diallylamino-[1,3,5]triazin-2-yl)piperazine

N-(diphenylmethyl)phenylcarbamate (75.7 mg, 0.25 mmol) and 1-(4,6-bis-diallyl amino-[1,3,5]triazin-2-yl)piperazine (88.7 mg, 0.25 mmol) were mixed in anhydrous THF (4 ml), and then DBU (37.3 μl, 0.25 mmol) was added dropwise to this reaction solution. After stirring for 19 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 116.4 mg (Yield 82.5%) of the title compound as a solid.

m.p.: 158-159° C.

¹H NMR (300 MHz, CDCl₃): δ ppm 7.16-7.35 (m, 10H), 6.16 (d, 1H, J=6.78 Hz), 5.75-5.88 (m, 4H), 5.12 (dd, 4H), 5.07 (dd, 4H), 4.98 (d, 1H), 4.12 (d, 8H), 3.78 (t, 4H), 3.42 (t, 4H)

### EXAMPLE 42

Synthesis of 1-[(diphenylmethyl)aminocarbonyl]-4-(4,6-bis-cyclopropylmethyl amino-[1,3,5]triazin-2-yl)piperazine

N-(diphenylmethyl)phenylcarbamate (51.6 mg, 0.17 mmol) and 1-(4,6-bis-cyclo propylmethylamino-[1,3,5]triazin-2-yl)piperazine (51.6 mg, 0.17 mmol) were mixed in anhydrous THF (4 ml), and then DBU (25.4 μl, 0.17 mmol) was added dropwise to this reaction solution. After stirring for 12 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 82.7 mg (Yield 94.9%) of the title compound as a solid.

m.p.: 186-188° C.

¹H NMR (300 MHz, CDCl₃): δ ppm 7.25-7.35 (m, 10H), 6.17 (d, 1H), 4.99 (d, 1H), 4.92 (s, 2H), 3.79 (s, 4H), 3.44 (dd, 4H), 3.21 (t, 4H), 0.99-1.05 (m, 2H), 0.30 (dt, 4H), 0.21 (dt, 4H)

### EXAMPLE 43

Synthesis of 1-[(2,2-diphenylethan-1-yl)amino-N-cyanocarboimidate]-4-(4,6-bis-allylamino-[1,3,5]triazin-2-yl)piperazine

N-(2,2-diphenylethan-1-yl)amino-phenoxy-N-cyanocarboimidate (155 mg, 0.45 mmol) and 1-(4,6-bis-allylamino-[1,3,5]triazin-2-yl)piperazine (125 mg, 0.45 mmol) were mixed in DMF (5 ml), and then 60% NaH (18.2 mg, 0.45 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=9:1, v/v) to give 65.9 mg (Yield 27.8%) of the title compound as a solid.

m.p.: 117-120° C.

¹H NMR (300 MHz, CDCl₃): δ ppm 7.26-7.37 (m, 10H), 5.85-5.94 (m, 2H), 5.20 (dd, 2H, J=17.22 Hz, J=1.47 Hz), 5.11 (dd, 2H, J=10.26 Hz, J=1.47 Hz), 4.90 (br, 2H, NH), 4.64 (t, 1H, J=5.28 Hz), 4.30 (t, 1H, J=8.04 Hz), 4.09 (dd, 2H, J=8.04 Hz, J=5.28 Hz), 3.98 (t, 4H), 3.69 (s, 4H), 3.26 (t, 4H)

### EXAMPLE 44

Synthesis of 1-[(diphenylmethyl)amino-N-cyanocarboimidate]-4-(4,6-bis-allylamino-[1,3,5]triazin-2-yl)piperazine

N-(diphenylmethyl)amino-phenoxy-N-cyanocarboimidate (142.8 mg, 0.44 mmol) and 1-(4,6-bis-allylamino-[1,3,5]triazin-2-yl)piperazine (120.1 mg, 0.44 mmol) were mixed in anhydrous DMF (5 ml), and then 60% NaH (17.4 mg, 0.44 mmol) was added dropwise to this reaction solution. After stirring for 6 hours at 80° C., the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:1, v/v) to give 135.7 mg (Yield 61.2%) of the title compound as a solid.

m.p.: 116-118° C.

¹H NMR (300 MHz, CDCl₃): δ ppm 7.24-7.34 (m, 10H), 6.22 (d, 1H, J=7.59 Hz), 5.83-5.93 (m, 2H), 5.77 (d, 1H, J=7.59 Hz), 5.18 (dd, 2H, J=17.04 Hz, J=1.47 Hz), 5.09 (dd, 2H, J=10.05 Hz, J=1.47 Hz), 5.07 (s, 2H), 3.97 (s, 4H), 3.78 (s, 4H), 3.50 (s, 4H)

### EXAMPLE 45

Synthesis of 1-[(2,2-diphenylethan-1-yl)aminocarbonyl]-4-(4,6-bis-allylamino-[1,3,5]triazin-2-yl)piperazine

N-(2,2-diphenylethan-1-yl)phenylcarbamate (66.1 mg, 0.22 mmol) and 1-(4,6-bis-allylamino-[1,3,5]triazin-2-yl)piperazine (60 mg, 0.22 mmol) were mixed in anhydrous THF (4 ml), and then DBU (32.6 μl, 0.22 mmol) was added dropwise to this reaction solution. After stirring for 19 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 81.8 mg (Yield 77.5%) of the title compound as a solid.

m.p. 90-92° C.

¹H NMR (300 MHz, CDCl₃): δ ppm 7.20-7.34 (m, 10H), 5.82-5.95 (m, 2H), 5.19 (dd, 2H), 5.09 (dd, 2H), 5.07 (br, 2H, NM, 4.41 (t, 1H, J=5.51 Hz), 4.23 (t, 1H, J=7.86 Hz), 3.98 (t, 4H, J=5.31 Hz), 3.88 (dd, 1H, J=7.86 Hz, J=5.51 Hz), 3.68 (s, 4H), 3.23 (t, 4H, J=5.31 Hz)

### EXAMPLE 46

Synthesis of 1-[5-ethyl-6-methyl-2-methoxypyridin-3-yl)aminocarbonyl]-4-(4,6-bis-allylamino-[1,3,5]triazin-2-yl)piperazine

N-(5-ethyl-6-methyl-2-methoxypyridin-3-yl)phenylcarbamate (50 mg, 0.17 mmol) and 1-(4,6-bis-allylamino-[1,3,5]triazin-2-yl)piperazine (48.1 mg, 0.17 mmol) were mixed in anhydrous THF (4 ml), and then DBU (26.1 μl, 0.17 mmol) was added dropwise to this reaction solution. After stirring for 4 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 27 mg (Yield 34%) of the title compound as a solid.

m.p.: 136-137° C.

¹H NMR (300 MHz, CDCl₃): δ ppm 8.19 (s, 1H), 6.88 (s, 1H), 5.90 (m, 2H), 5.22 (dd, 2H), 5.12 (dd, 2H), 4.02 (t, 4H), 3.97 (s, 3H), 3.86 (s, 4H), 3.54 (t, 4H), 2.52 (q, 2H, J=0.5 Hz), 2.37 (s, 3H), 1. 17 (t, 3H, J=0.5 Hz)

¹³C NMR (75 MHz, CDCl₃): δ ppm 164.99, 154.68, 150.13, 144.81, 135.10, 130.05, 126.50, 121.07, 115.74, 53.41, 43.62, 43.19, 42.68, 29.67, 25.26, 20.85, 14.57

### EXAMPLE 47

Synthesis of 1-[(5-ethyl-6-methyl-2-methoxypyridin-3-yl)aminocarbonyl]-4-[(2,6-dipyrrolidin-1-yl)pyrimidin-4-yl]piperazine

N-(5-ethyl-6-methyl-2-methoxypyridin-3-yl)phenylcarbamate (44.3 mg, 0.15 mmol) and 1-[(2,6-dipyrrolidin-1-yl)pyrimidin-4-yl]piperazine (46.9 mg, 0.15 mmol) were mixed in anhydrous THF (4 ml), and then DBU (23 μl, 0.15 mmol) was added dropwise to this reaction solution. After stirring for 2 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:2, v/v) to give 10.2 mg (Yield 13.7%) of the title compound as a solid.

m.p. 162-166° C.

¹H NMR (300 MHz, CDCl₃): δ ppm 8.20 (s, 1H), 6.89 (s, 1H), 4.86 (s, 1H), 3.97 (s, 3H), 3.43-3.66 (m, 16H), 2.55 (q, 2H, J=7.5 Hz), 2.33 (s, 3H), 1.91 (dd, 8H, J=6.42 Hz, J=2.37 Hz), 1.17 (t, 3H, J=7.5 Hz)

### EXAMPLE 48

Synthesis of 1-[N-(2-methoxyquinoxalin-3-yl)aminocarbonyl]-4-(diphenyl methyl)piperazine

N-(2-methoxyquinoxalin-3-yl)phenylcarbamate (50 mg, 0.17 mmol) and 1-(diphenylmethyl)piperazine (42.7 mg, 0.17 mmol) were mixed in anhydrous THF (5 ml), and then DBU (28 μl, 0. 19 mmol) was added dropwise to this reaction solution. After stirring for 1 hour at room temperature, the product was extracted with methylene chloride (50 ml ×3). The organic solution was washed with aqueous sodium chloride solution and water, dried over anhydrous magnesium sulfate, and concentrated under reduced pressure. The residue was crystallized from ethyl acetate and diethylether to give 43.1 mg (Yield 56.2%) of the title compound as a solid.

¹H NMR (300 MHz, CDCl₃): δ ppm 14.33 (s, 1H), 7.19-7.84 (m, 4H), 4.10 (s, 3H), 4.07 (s, 1H), 3.64 (d, 4H, J=18.84 Hz), 2.46 (d, 4H, J=18.84 Hz)

### EXAMPLE 49

Synthesis of 1-[N-(2-ethoxyquinoxalin-3-yl)aminocarbonyl]-4-(diphenyl methyl)piperazine

N-(2-ethoxyquinoxalin-3-yl)phenylcarbamate (100 mg, 0.32 mmol) and 1-(diphenyl methyl)piperazine (80.7 mg, 0.32 mmol) were mixed in anhydrous THF (2 ml), and then DBU (70 μl, 0.45 mmol) was added dropwise to this reaction solution. After stirring for 3.5 hours at room temperature, the product was extracted with methylene chloride (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane:methylene chloride:methanol=100:150:100:2.5, v/v) to give 73.6 mg (Yield 49.3%) of the title compound as a solid.

m.p.: 156-158° C.

¹H NMR (500 MHz, CDCl₃): δ ppm 7.19-7.79 (m, 15H), 4.58 (q, 2H), 4.31 (s, 1H), 3.61 (m, 4H), 2.50 (m, 4H), 1.46 (t, 3H)

### EXAMPLE 50

Synthesis of 1-[N-(2-phenoxyquinoxalin-3-yl)aminocarbonyl]-4-(diphenyl methyl)piperazine

N-(2-phenoxyquinoxalin-3-yl)phenylcarbamate (50 mg, 0.14 mmol) and 1-(diphenyl methyl)piperazine (35.3 mg, 0.14 mmol) were mixed in anhydrous THF (0.7 ml), and then DBU (30 μl, 0.19 mmol) was added dropwise to this reaction solution. After stirring for 2 hours at room temperature, the product was extracted with methylene chloride (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane:methylene chloride:methanol=20:30:20:0.5, v/v) to give 52.1 mg (Yield 72.4%) of the title compound as a solid.

m.p.: 208-210° C.

¹H NMR (500 MHz, CDCl₃): δ ppm 7.19-7.84 (m, 20H), 4.32 (s, 1H), 4.00 (s, 1H), 3.66 (m, 4H), 2.52 (m, 4H)

### EXAMPLE 51

Synthesis of 1-[N-(2-methoxyquinoxalin-3-yl)aminocarbonyl]-4-(4,6-bis-allyl amino-[1,3,5]triazin-2-yl)piperazine

N-(2-methoxyquinoxalin-3-yl)phenylcarbamate (80 mg, 0.27 mmol) and 1-(4,6-bis-allylamino-[1,3,5]triazin-2-yl)piperazine (74.6 mg, 0.27 mmol) were mixed in anhydrous THF (3 ml), and then DBU (40.5 μl, 0.27 mmol) was added dropwise to this reaction solution. After stirring for 3.5 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=4:1, v/v) to give 91.4 mg (Yield 71.7%) of the title compound as a solid.

m.p.: 158-159° C.

¹H NMR (500 MHz, CDCl₃): δ ppm 7.21-7.82 (m, 4H), 5.91 (m, 2H), 5.21 (d, 2H), 5.11 (d, 2H), 4.93 (s, 2X, NH), 4.15 (d, 3H), 3.83-4.01 (m, 8H), 3.65 (t, 4H)

### EXAMPLE 52

Synthesis of 1-[N-(2-methoxyquinoxalin-3-yl)aminocarbonyl]-4-{4,6-bis-[ethyl -(2-methylallyl)amino]-[1,3,5]triazin-2-yl}piperazine

N-(2-methoxyquinoxalin-3-yl)phenylcarbamate (79.2 mg, 0.27 mmol) and 1-{4,6-bis-[ethyl-(2-methylallyl)amino]-[1,3,5]triazin-2-yl}piperazine (96.4 mg, 0.27 mmol) were mixed in anhydrous THF (3 ml), and then DBU (40.1 μl, 0.27 mmol) was added dropwise to this reaction solution. After stirring for 3 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 63.8 mg (Yield 42.2%) of the title compound as a solid.

m.p. 150-152° C.

¹H NMR (500 Mz, CDCl₃): δ ppm 7.49-7.84 (m, 4H), 4.80 (s, 4H), 4.15 (s, 3H), 4.15 (s, 4H), 3.91 (m, 4H), 3.63 (s, 4H), 3.50 (s, 4H), 1.69 (s, 6H), 1.12 (t, 6H)

### EXAMPLE 53

Synthesis of 1-[N-(2-methoxyquinoxalin-3-yl)aminocarbonyll-4-(4,6-bis-diallylamino-[1,3,5]triazin-2-yl)piperazine

N-(2-methoxyquinoxalin-3 -yl)phenylcarbamate (84.5 mg, 0.29 mmol) and 1-(4,6-bis-diallylamino-[1,3,5]triazin-2-yl)piperazine (101.7 mg, 0.29 mmol) were mixed in anhydrous THF (4 ml), and then DBU (42.8/9, 0.29 mmol) was added dropwise to this reaction solution. After stirring for 3.5 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:4, v/v) to give 111.7 mg (Yield 69.2%) of the title compound as a solid.

m.p.: 143-145° C.

¹H NMR (500 MHz, CDCl₃): δ ppm 7.69 (br, 2H), 7.42 (br, 2H), 5.80-5.83 (m, 4H), 5.12 (d, 4H), 5. 10 (d, 4H), 4.14 (m, 11H), 3.87 (s, 4H), 3.66 (s, 4H)

### EXAMPLE 54

Synthesis of 1-[(5-fluoro-1H-pyrimidin-2,4-dioxo-1-yl)carbonyl]-4-(3,5-dimethylphenyl)piperazine

N-(5-fluoro-1H-pyrimidin-2,4-dioxo-1-yl)phenylcarbamate (227.9 mg, 1.13 mmol) and 1-(3,5-dimethylphenyl)piperazine (215 mg, 1.13 mmol) were mixed in anhydrous THF (20 ml), and then DBU (0.22 ml, 1.35 mmol) was added dropwise to this reaction solution. After stirring for 2 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:10, v/v) to give 261.4 mg (Yield 68.0%) of the title compound as a solid.

m.p.: 68-72° C.

¹H NMR (300 MHz, CDCl₃): δ ppm 6.74 (d, 1H, J=7.5 Hz), 6.59 (s, 3H), 3.76 (d, 4H), 3.20 (t, 4H, J=4.95 Hz, J=5.13 Hz), 2.29 (s, 6H)

### EXAMPLE 55

Synthesis of 1-[(1H-imidazol-1-yl)carbonyl]-4-(3,5-dimethylphenyl)piperazine 1,1′-Carbonyldiimidazole (81 mg, 0.53 mmol) and 1-(3,5-dimethylphenyl) piperazine (105 mg, 0.53 mmol) were mixed in anhydrous THF (10 ml), and then DBU (80 μl, 0.53 mmol) was added dropwise to this reaction solution. After stirring for 6 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:1, v/v) to give 69 mg (Yield 48.3%) of the title compound.

¹H NMR (300 MHz, CDCl₃): δ ppm 7.90 (s, 1H), 7.23 (s, 1H), 7.11 (s, 1H), 6.52 (m, 3H), 3.75, 3.22 (2m, 8H), 2.26 (s, 6H)

### EXAMPLE 56

Synthesis of 1-[(1H-imidazol-1-yl)thiocarbonyl]-4-(3,5-dimethylphenyl) piperazine

1,1′-Thiocarbonyldiimidazole (51.4 mg, 0.29 mmol) and 1-(3,5-dimethylphenyl) piperazine (54.9 mg, 0.29 mmol) were mixed in anhydrous THF (10 ml), and then DBU (43 μl, 0.29 mmol) was added dropwise to this reaction solution. After stirring for 6 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:1, v/v) to give 59.5 mg (Yield 68.6%) of the title compound.

m.p.: 103-105° C.

¹H NMR (300 MHz, CDCl₃): δ ppm 7.90 (s, 1H), 7.24 (s, 1H), 7.11 (s, 1H), 6.56 (m, 3H), 4.07, 3.29 (2m, 8H), 2.29 (s, 6H)

### EXAMPLE 57

Synthesis of 1-[(1H-imidazol-1-yl)carbonyl]-4-(3,5-dimethylphenyl)piperazine hydrochloride

1-[(1H-imidazol-1-yl)carbonyl]-4-(3,5-dimethylphenyl)piperazine (39.8 mg, 0.13 mmol) prepared in Example 55 was dissolved in anhydrous methylene chloride (10 ml) and the resulting solution was saturated with HCl gas for 2 hours at 0° C. The saturated solution was allowed to stand for 2 days at 4° C. and the resulting yellow solid was filtered to give 35 mg (Yield 78.7%) of the title compound.

m.p.: 170-172° C.

¹H NMR (500 MHz, DMSO): δ ppm 9.70 (s, 1H), 8.14 (s, 1H), 7.88 (s, 1H), 7.12 (s, 2H), 6.86 (s, 1H), 3.90 (s, 4H), 3.55 (s, 4H), 2.31 (6H, Me)

¹³C NMR (125.8 MHz, CDCl₃+DMSO): δ ppm 147.14, 138.76, 138.68, 136.51, 120.85, 120.22, 116.37, 50.47, 44.51, 21.10, 21.03

EI MS, m/e=320

### EXAMPLE 58

Synthesis of 1-[(1H-imidazol-1-yl)carbonyl]-4-(3,5-dimethoxyphenyl) piperazine

1,1′-Carbonyldiimidazole (41 mg, 0.25 mmol) and 1-(3,5-dimethoxyphenyl) piperazine (55.5 mg, 0.25 mmol) were mixed in anhydrous THF (10 ml), and then DBU (48 μl, 0.32 mmol) was added dropwise to this reaction solution. After stirring for 3 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:methylene chloride:methanol=20:1, v/v) to give 46 mg (Yield 58.2%) of the title compound.

¹H NMR (300 MHz, CDCl₃): δ ppm 7.96 (s, 1H), 7.23 (s, 1H), 7.11 (s, 1H), 6.08 (m, 3H), 3.75 (s, 6H), 3.73, 3.23 (2m, 8H)

### EXAMPLE 59

Synthesis of 1-[(1H-imidazol-1-yl)carbonyl]-4-(4,6-bis-allylamino-[1,3,5]triazin-2-yl)piperazine

1,1′-Carbonyldiimidazole (25.5 mg, 0.16 mmol) and 1-(4,6-bis-allylamino-[1,3,5]triazin-2-yl)piperazine (43.3 mg, 0.16 mmol) were mixed in anhydrous THF (4 ml), and then DBU (23.9 μl, 0.16 mmol) was added dropwise to this reaction solution. After stirring for 12 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=4: 1, v/v) to give 37.1 mg (Yield 63.9%) of the title compound.

m.p.: 78-82° C.

¹H NMR (300 MHz, CDCl₃): δ ppm 7.91 (s, 1H), 7.23 (s, 1H), 7.12 (s, 1H), 5.84-5.97 (m, 2H), 5.21 (dd, 2H, J=17.1 Hz, J=1.47 Hz), 5.10-5.13 (s, 2H), 5.11 (dd, 2H, J=10.26 Hz, J=1.47 Hz), 4.00 (s, 4H), 3.87 (s, 4H), 3.63 (t, 4H)

### EXAMPLE 60

Synthesis of 1-[(1H-imidazol-1-yl)thiocarbonyl]-4-(4,6-bis-allylamino-[1,3,5]triazin-2-yl)piperazine

1,1′-Thiocarbonyldiimidazole (36.2 mg, 0.2 mmol) and 1-(4,6-bis-allylamino-[1,3,5] triazin-2-yl)piperazine (55.9 mg, 0.2 mmol) were mixed in anhydrous THF (4 ml), and then DBU (30.3 μl, 0.2 mmol) was added dropwise to this reaction solution. After stirring for 12 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=4:1, v/v) to give 35.6 mg (Yield 45.5%) of the title compound.

m.p.: 124-127° C.

¹H NMR (300 MHz, CDCl₃): δ ppm 7.90 (s, 1H), 7.22 (s, 1H), 7.11 (s, 1H), 5.84-5.96 (m, 2H), 5.09-5.24 (m, 2H), 5.20 (dd, 21H, J=17.13 Hz, J=1.29 Hz), 5.11 (dd, 2H, J=10.17 Hz, J=1.29 Hz), 3.92-4.00 (m, 12H)

### EXAMPLE 61

Synthesis of 1-[(1H-imidazol-1-yl)carbonyl]-4-(4,6-bis-diallylamino-[1,3,5]triazin-2-yl)piperazine

1,1′-Carbonyldiimidazole (34.0 mg, 0.23 mmol) and 1-(4,6-bis-diallylamino-[1,3,5]triazin-2-yl)piperazine (74.6 mg, 0.21 mmol) were mixed in anhydrous THF (2 ml), and then DBU (34.5 μl, 0.23 mmol) was added dropwise to this reaction solution. After stirring for 29 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 74.5 mg (Yield 79%) of the title compound.

m.p.: 94-95° C.

¹H NMR (300 MHz, CDCl₃): δ ppm 7.89 (s, 1H), 7.21 (s, 1H), 7.11 (s, 1H), 5.75-5.88 (m, 4H), 5.13 (dd, 4H, J=8.43 Hz, J=1.47 Hz), 5.09 (s, 4H), 4.13 (d, 8H, J=5.67 Hz), 3.86 (t, 4H), 3.61 (t, 4H)

¹³C NMR (125.8 MHz, CDCl₃): δ ppm 165.49, 165.26, 151.07, 136.93, 134.42, 129.88, 117.90, 116.29, 48.38, 46.43, 42.93

EI MS, m/e=449

### EXAMPLE 62

Synthesis of 1-[(1H-imidazol-1-yl)carbonyl]-4-(4,6-bis-cyclopropylmethyl amino-[1,3,5]triazin-2-yl)piperazine

1,1′-Carbonyldiimidazole (35.3 mg, 0.22 mmol) and 1-(4,6-bis-cyclopropylmethylamino-[1,3,5]triazin-2-yl)piperazine (60 mg, 0.2 mmol) were mixed in anhydrous THF (4 ml), and then DBU (29.6 μl, 0.2 mmol) was added dropwise to this reaction solution. After stirring for 16 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=9:1, v/v) to give 59.6 mg (Yield 75.0%) of the title compound.

m.p.: 132-133° C.

¹H NMR (500 MHz, CDCl₃): δ ppm 7.90 (s, 1H), 7.23 (s, 1H), 7.12 (s, 1H), 4.95 (s, 2H), 3.87 (s, 4H), 3.21 (dd, 4H), 0.93-1.06 (m, 2H), 0.50 (dt, 4H), 0.23 (dt, 4H)

¹³C NMR (125.8 MHz, CDCl₃): δ ppm 166.10, 165.22, 151.03, 136.94, 129.90, 117.92, 46.44, 45.69, 42.77, 10.99, 3.36

EI MS, m/e=397

### EXAMPLE 63

Synthesis of 1-[(1H-imidazol-1-yl)carbonyl]-4-{4,6-bis-[ethyl-(2-methylallyl) amino]-[1,3,5]triazin-2-yl}piperazine

1,1′-Carbonyldiimidazole (3 5.9 mg, 0.24 mmol) and 1-{4,6-bis-[ethyl-(2-methylallyl)amino]-[1,3,5]triazin-2-yl}piperazine (79.5 mg, 0.22 mmol) were mixed in anhydrous THF (4 ml), and then DBU (33.1 μl, 0.22 mmol) was added dropwise to this reaction solution. After stirring for 15.5 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 84.9 mg (Yield 84.7%) of the title compound.

¹H NMR (300 MHz, CDCl₃): δ ppm 7.90 (s, 1H), 7.23 (s, 1H), 7.11 (s, 1H), 4.79 (d, 4H), 4.11 (s, 4H), 3.86 (s, 4H), 3.63 (s, 4H), 3.49 (s, 4H), 1.68 (s, 6H), 0.90 (t, 6H)

### EXAMPLE 64

Synthesis of 1-[(1H-imidazol-1-yl)carbonyl]-4-[4,6-bis-(allyl-cyclohexylamino)-[1,3,5]triazin-2-yl]piperazine

1,1′-Carbonyldiimidazole (20.9 mg, 0.13 mmol) and 1-[4,6-bis-(allyl-cyclohexylamino)-[1,3,5]triazin-2-yl]piperazine (51.5 mg, 0.12 mmol) were mixed in anhydrous THF (4 ml), and then DBU (17.5 μl, 0.12 mmol) was added dropwise to this reaction solution. After stirring for 15 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:1, v/v) to give 50.5 mg (Yield 80.8%) of the title compound.

m.p.: 132-135° C.

¹H NMR (300 MHz, CDCl₃): δ ppm 7.90 (s, 1H), 7.23 (s, 1H), 7.12 (s, 1H), 5.87 (m, 2H), 5.12 (d, 2H, J=17.4 Hz), 5.02 (d, 2H, J=10.08 Hz), 4.44 (s, 2H), 4.06 (d, 4H), 3.84 (s, 4H), 3.62 (s, 4H), 1.09-1.78 (m, 10H)

### EXAMPLE 65

Synthesis of 1-[(1H-imidazol-1-yl)carbonyl]-4-{4,6-bis-(2,5-dihydropyrrol-1-yl)-[1,3,5]triazin-2-yl}piperazine

1,1′-Carbonyldiimidazole (30.6 mg, 0.19 mmol) and 1-{4,6-bis-(2,5-dihydropyrrol-1-yl)-[1,3,5]triazin-2-yl}piperazine (51.4 mg, 0.17 mmol) were mixed in anhydrous THF (4 ml), and then DBU (25.7 μl, 0.17 mmol) was added dropwise to this reaction solution. After stirring for 18 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=2:1, v/v) to give 64.0 mg (Yield 94.8%) of the title compound.

m.p.: 160-162° C.

¹H NMR (300 MHz, CDCl₃): δ ppm 7.91 (s, 1H), 7.23 (s, 1H), 7.12 (s, 1H), 5.87 (t, 4H), 4.29 (d, 8H, J=2.73 Hz), 3.91 (t, 4H), 3.64 (t, 4H)

### EXAMPLE 66

Synthesis of 3-[(1H-imidazol-1-yl)carbonyl]-7,8-dimethoxy-1-phenyl-2,3,4,5-tetrahydro-3H-benzazepine

1,1′-Carbonyldiimidazole (1.75 g, 10.8 mmol) and 7,8-dimethoxy-1-phenyl-2,3,4,5-tetrahydro-3H-benzazepine (3.06 g, 10.8 mmol) were mixed in anhydrous THF (40 ml), and then DBU (1.8 ml, 11.9 mmol) was added dropwise to this reaction solution. After stirring for 12 hours at room temperature, the product was extracted with ethyl acetate (100 ml×3). The organic solution was washed with aqueous sodium chloride solution and water, dried over anhydrous magnesium sulfate, and concentrated under reduced pressure. The residue was separated by column chromatography (eluent:ethyl acetate:hexane=10:1, v/v) to give 3.01 g (Yield 73.8%) of the title compound.

m.p.: 132-133° C.

¹H NMR (500 MHz, CDCl₃): δ ppm 7.63 (s, 1H), 7.23-7.32 (m, 4H), 7.02-7.03 (m, 4H), 6.66 (s, 1H), 6.55 (s, 1H), 4.62 (s, 1H), 4.08-4.13 (m, 2H), 3.89 (s, 3H), 3.75-3.78 (m, 1H), 3.76 (s, 3H), 3.60 (m, 1H), 3.23 (m, 1H), 2.86 (d, 1H)

¹³C NMR (125.8 MHz, CDCl₃): δ ppm 151.85, 148.03, 147.71, 136.75, 131.80, 129.52, 128.86, 127.72, 126.95, 117.88, 114.56, 113.62, 56.00, 55.91, 51.37, 50.53, 49.62, 34.01

EI MS, m/e=377

### EXAMPLE 67

Synthesis of 3-[(1H-imidazol-1-yl)thiocarbonyl]-7,8-dimethoxy-1-phenyl-2,3,4,5-tetrahydro-3H-benzazepine

1,1′-Thiocarbonyldiimidazole (50.3 mg, 0.28 mmol) and 7,8-dimethoxy-1-phenyl-2,3,4,5-tetrahydro-3H-benzazepine (80 mg, 0.28 mmol) were mixed in anhydrous THF (10 ml), and then DBU (42 μl, 0.28 mmol) was added dropwise to this reaction solution. After stirring for 12 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:9, v/v) to give 75.5 mg (Yield 68.5%) of the title compound.

m.p.: 124-125° C.

¹H NMR (300 MHz, CDCl₃): δ ppm 7.55 (s, 1H), 7.22-7.33 (m, 4H), 6.97-7.02 (m, 4H), 6.63 (s, 1H), 6.55 (s, 1H), 4.20 (dd, 1H, J=13.7 Hz), 3.67-3.88 (m, 3H), 3.88 (s, 3H), 3.75 (s, 3H), 3.31 (s, 1H), 2.88 (s, 1H)

### EXAMPLE 68

Synthesis of 3-[(1H-imidazol-1-yl)carbonyl]-(R)-7,8-dimethoxy-1-phenyl-2,3,4,5-tetrahydro-3H-benzazepine

1,1′-Carbonyldiimidazole (133.9 mg, 0.56 mmol) and (R)-7,8-dimethoxy-1-phenyl-2,3,4,5-tetrahydro-3H-benzazepine (153.7 mg, 0.56 mmol) were mixed in anhydrous THF (3 ml), and then DBU (84.3 μl, 0.56 mmol) was added dropwise to this reaction solution. After stirring for 18 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=10:1, v/v) to give 170 mg (Yield 79.8%) of the title compound.

m.p.: 112-114° C.

¹H NMR (500 MHz, CDCl₃): δ ppm 7.63 (s, 1H), 7.23-7.32 (m, 4H), 7.02-7.03 (m, 4H), 6.66 (s, 1H), 6.55 (s, 1H), 4.62 (s, 1H), 4.08-4.13 (m, 2H), 3.89 (s, 3H), 3.75-3.78 (m, 1H), 3.76 (s, 3H), 3.60 (m, 1H), 3.23 (m, 1H), 2.86 (d, 1H)

¹³C NMR (125.8 MHz, CDCl₃): δ ppm 151.85, 148.03, 147.71, 136.75, 131.80, 129.52, 128.86, 127.72, 126.95, 117.88, 114.56, 113.62, 56.00, 55.91, 51.37, 50.53, 49.62, 34.01

EI MS, m/e=377

### EXAMPLE 69

Synthesis of 3-[(1H-imidazol-1-yl)carbonyl]-(S)-7,8-dimethoxy-1-phenyl-2,3,4,5-tetrahydro-3H-benzazepine

1,1′-Carbonyldiimidazole (48.1 mg, 0.20 mmol) and (S)-7,8-dimethoxy-1-phenyl-2,3,4,5-tetrahydro-3H-benzazepine (55.2 mg, 0.20 mmol) were mixed in anhydrous THF (3 ml), and then DBU (30.3 μl, 0.20 mmol) was added dropwise to this reaction solution. After stirring for 20 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:ethyl acetate:hexane=10:1, v/v) to give 52.3 mg (Yield 69.3%) of the title compound.

m.p.: 131-132° C.

¹H NMR (300 MHz, CDCl₃): δ ppm 7.63 (s, 1H), 7.23-7.32 (m, 4H), 7.02-7.03 (m, 4H), 6.66 (s, 1H), 6.55 (s, 1H), 4.62 (s, 1H), 4.08-4.13 (m, 2H), 3.89 (s, 3H), 3.75-3.78 (m, 1H), 3.76 (s, 3H), 3.60 (m, 1H), 3.23 (m, 1H), 2.86 (d, 1H)

### EXAMPLE 70

Synthesis of 3-[(1H-imidazol-1-yl)carbonyl]-7,8-dimethoxy-1-(4-fluoro)phenyl-2,3,4,5-tetrahydro-3H-benzazepine

1,1′-Carbonyldiimidazole (27.1 mg, 0.17 mmol) and 7,8-dimethoxy-1-(4-fluoro)phenyl-2,3,4,5-tetrahydro-3H-benzazepine (50.3 mg, 0.17 mmol) were mixed in anhydrous THF (2 ml), and then DBU (25 μl, 0.17 mmol) was added dropwise to this reaction solution. After stirring for 22 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:methylene chloride:methanol=20:1, v/v) to give 35.1 mg (Yield 53.2%) of the title compound.

m.p.: 145-147° C.

¹H NMR (500 MHz, CDCl₃): δ ppm 7.68 (s, 1H), 6.98-7.05 (m, 6H), 6.65 (s, 1H), 6.52 (s, 1H), 4.60 (t, 1H), 4.04-4.13 (m, 2H), 3.89 (s, 3H), 3.75-3.77 (m, 4H), 3.62 (m, 1H), 3.20 (m, 1H), 2.84 (m, 1H)

¹³C NMR (125.8 MHz, CDCl₃): δ ppm 162.60, 160.85, 151.77, 148.02, 147.71, 137.90, 136.87, 131.56, 129.56, 129.23, 129.17, 117.83, 115.72, 115.55, 114.34, 113.57, 55.93, 55.86, 50.89, 49.75, 49.58, 33.95

EI MS, m/e=395

### EXAMPLE 71

Synthesis of 3-[(1H-imidazol-1-yl)carbonyl]-7,8-dimethoxy-1-(4-chloro)phenyl-2,3,4,5-tetrahydro-3H-benzazepine

1,1′-Carbonyldiimidazole (30.2 mg, 0.19 mmol) and 7,8-dimethoxy-1-(4-chloro) phenyl-2,3,4,5-tetrahydro-3H-benzazepine (59.1 mg, 0.19 mmol) were mixed in anhydrous THF (3 ml), and then DBU (27.8 μl, 0.19 mmol) was added dropwise to this reaction solution. After stirring for 16 hours at room temperature, the product was extracted with ethyl acetate (30 ml×3). The organic solution was washed with aqueous sodium chloride solution and water, dried over anhydrous magnesium sulfate, and concentrated under reduced pressure. The residue was separated by column chromatography (eluent:methylene chloride:methanol=20:1, v/v) to give 49 mg (Yield 64.0%) of the title compound.

m.p.: 147-149° C.

¹H NMR (500 MHz, CDCl₃): δ ppm 7.68 (s, 1H), 6.96-7.28 (m, 6H), 6.65 (s, 1H), 6.53 (s, 1H), 4.61 (t, 1H), 4.07 (m, 2H), 4.03 (s, 3H), 3.77 (m, 4H), 3.58 (m, 1H), 3.19 (m, 1H), 2.83 (m, 1H)

¹³C NMR (125.8 MHz, CDCl₃): δ ppm 151.67, 147.96, 147.68, 136.60, 132.62, 131.09, 129.46, 128.96, 128.82, 117.76, 114.27, 113.51, 55.84, 55.80, 50.89, 49.76, 49.58, 33.81

EI MS, m/e=411

### EXAMPLE 72

Synthesis of 3-[(1H-imidazol-1-yl)carbonyl]-8-methoxy-1-phenyl-2,3,4,5-tetrahydro-3H-benzazepine

1,1′-Carbonyldiimidazole (105.7 mg, 0.65 mmol) and 8-methoxy-1-phenyl-2,3,4,5-tetrahydro-3H-benzazepine (150.1 mg, 0.59 mmol) were mixed in anhydrous THF (4 ml), and then DBU (88.6μl, 0.59 mmol) was added dropwise to this reaction solution. After stirring for 18 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=9:1, v/v) to give 173.1 mg (Yield 84.5%) of the title compound.

m.p.: 65-68° C.

¹H NMR (500 MHz, CDCl₃): δ ppm 7.63 (s, 1H), 7.24-7.30 (m, 3H), 7.02-7.10 (m, 5H), 6.76 (d, 1H), 6.59 (s, 1H), 4.61 (s, 1H), 4.10 (m, 2H), 3.73 (m, 4H), 3.59 (m, 1H), 3.18 (m, 1H), 2.91 (m, 1H)

EI MS, m/e=347

### EXAMPLE 73

Synthesis of 3-[(1H-imidazol-1-yl)carbonyl]-8-methoxy-1-(4-fluoro)phenyl-2,3,4,5-tetrahydro-3H-benzazepine

1,1′-Carbonyldiimidazole (60.6 mg, 0.37 mmol) and 8-methoxy-1-(4-fluoro)phenyl-2,3,4,5-tetrahydro-3H-benzazepine (92.6 mg, 0.34 mmol) were mixed in anhydrous THF (3 ml), and then DBU (50.8 μl, 0.34 mmol) was added dropwise to this reaction solution. After stirring for 12 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=4:1, v/v) to give 79.6 mg (Yield 63.9%) of the title compound.

m.p. 65-69° C.

¹H NMR (500 MHz, CDCl₃): δ ppm 7.67 (s, 1H), 6.99-7.10 (m, 7H), 6.76 (d, 1H), 6.56 (s, 1H), 4.60 (s, 1H), 4.07 (m, 2H), 3.74 (m, 4H), 3.59 (m, 1H), 3.14 (m, 1H), 2.01 (m, 1H)

### EXAMPLE 74

Synthesis of 3-[(1H-imidazol-1-yl)carbonyl]-8-methoxy-1-(4-chloro)phenyl-2,3,4,5-tetrahydro-3H-benzazepine

1,1′-Carbonyldiimidazole (99.6 mg, 0.61 mmol) and 8-methoxy-1-(4-chloro)phenyl-2,3,4,5-tetrahydro-3H-benzazepine (176.8 mg, 0.61 mmol) were mixed in anhydrous THF (3 ml), and then DBU (91.8 μl, 0.61 mmol) was added dropwise to this reaction solution. After stirring for 16 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=9:1, v/v) to give 170 mg (Yield 79.8%) of the title compound.

m.p.: 66-70° C.

¹H NMR (500 MHz, CDCl₃): δ ppm 7.68 (t, 1H), 7.25-7.29 (m, 2H), 6.98-7.10 (m, 5H), 6.76 (dd, 1H, J=2.68 Hz, J=8.24 Hz), 6.56 (d, 1H, J=2.68 Hz), 4.60 (t, 1H, 4.07 (m, 2H), 3.74 (m, 4H), 3.60 (m, 1H), 3.14 (m, 1H), 2.90 (m, 1H)

### EXAMPLE 75

Synthesis of 3-[(1H-imidazol-1-yl)carbonyl]-1-phenyl-2,3,4,5-tetrahydro-3H-benzazepine

1,1′-Carbonyldiimidazole (79.1 mg, 0.49 mmol) and 1-phenyl-2,3,4,5-tetrahydro-3H-benzazepine (109.0 mg, 0.49 mmol) were mixed in anhydrous THF (3 ml), and then DBU (73 μl, 0.49 mmol) was added dropwise to this reaction solution. After stirring for 21 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=9:1, v/v) to give 150.9 mg (Yield 97.1%) of the title compound.

m.p.: 98-99° C.

¹H NMR (300 MHz, CDCl₃): δ ppm 7.62 (s, 1H), 7.18-7.32 (m, 6H), 7.01-7.03 (m, 5H), 4.67 (t, 1H), 4.06-4.20 (m, 2H), 3.76 (m, 1H), 3.62 (m, 1H), 3.25 (m, 1H), 2.96 (m, 1H)

### EXAMPLE 76

Synthesis of 3-[(1H-imidazol-1-yl)carbonyl]-1-(4-fluoro)phenyl-2,3,4,5-tetrahydro-3H-benzazepine

1,1′-Carbonyldiimidazole (82.4 mg, 0.51 mmol) and 1-(4-fluoro)phenyl-2,3,4,5-tetra hydro-3H-benzazepine (112 mg, 0.46 mmol) were mixed in anhydrous THF (3ml), and then DBU (69 μl, 0.46 mmol) was added dropwise to this reaction solution. After stirring for 17 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=9:1, v/v) to give 147.7 mg (Yield 95.5%) of the title compound.

m.p.: 65-68° C.

¹H NMR (300 MHz, CDCl₃): δ ppm 7.65 (s, 1H), 7.19-7.26 (m, 3H), 7.00-7.04 (m, 7H), 4.65 (s, 1H), 4.09 (m, 2H), 3.76 (m, 1H), 3.61 (m, 1H), 3.21 (m, 1H), 2.95 (m, 1H)

### EXAMPLE 77

Synthesis of 3-[(1H-imidazol-1-yl)carbonyl]-1-(4-chloro)phenyl-2,3,4,5-tetrahydro-3H-benzazepine

1,1′-Carbonyldiimidazole (47.9 mg, 0.30 mmol) and 1-(4-chloro)phenyl-2,3,4,5-tetrahydro-3H-benzazepine (76.1 mg, 0.30 mmol) were mixed in anhydrous THF (3 ml), and then DBU (44 μl, 0.30 mmol) was added dropwise to this reaction solution. After stirring for 19 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:methylene chloride:methanol=20:1, v/v) to give 89.5 mg (Yield 86.2%) of the title compound.

m.p.: 60-64° C.

¹H NMR (300 MHz, CDCl₃): δ ppm 7.66 (s, 1H), 6.96-7.28 (m, 10H), 4.65 (s, 1H), 4.10 (m, 2H), 3.78 (m, 1H), 3.61 (m, 1H), 3.20 (m, 1H), 2.94 (m, 1H)

### EXAMPLE 78

Synthesis of 3-[(1H-imidazol-1-yl)carbonyl]-7,8-dihydroxy-1-phenyl-2,3,4,5-tetrahydro-3H-benzazepine

3-[(1H-imidazol-1-yl)carbonyl]-7,8-dimethoxy-1 -phenyl-2,3,4,5-tetrahydro-3H-benzazepine (200 mg, 0.53 mmol) prepared in Example 66 was dissolved in anhydrous methylene chloride (1.8 ml) and the resulting solution was slowly added dropwise to BBr₃ (0.25 ml, 2.65 mmol) dissolved in anhydrous methylene chloride (0.8 ml) at 15° C. The reaction solution was stirred for 3 hours at 25° C., the temperature was cooled to −30° C., and then methanol (1.6 ml) was added to the reaction mixture. The organic solution was concentrated under reduced pressure and the residue was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:methylene chloride:methanol=10:1, v/v) to give 177.6 mg (Yield 96%) of the title compound.

m.p.: 148-150° C.

¹H NMR (500 MHz, CDCl₃): δ ppm 7.62 (s, 1H), 7.21-7.26 (m, 3H), 6.99-7.03 (m, 4H), 6.67 (s, 1H), 6.53 (s, 1H), 4.52 (s, 1H), 4.11 (m, 2H), 3.92 (m, 1H), 3.66 (2H, OH), 3.56 (m, 1H), 3.12 (m, 1H), 2.78 (m, 1H)

### EXAMPLE 79

Synthesis of 3-[(1H-imidazol-1-yl)carbonyl]-7,8-diethoxy-1-phenyl-2,3,4,5-tetrahydro-3H-benzazepine

3-[(1H-imidazol-1-yl)carbonyl]-7,8-dihydroxy-1-phenyl-2,3,4,5-tetrahydro-3H-benzazepine (90 mg, 0.26 mmol) prepared in Example 78 and ethyl iodide (43.3 μl, 0.54 mmol) were mixed in anhydrous DMF (3 ml), and then NaH (60%, 21.7 mg, 0.54 mmol) was added dropwise to this reaction solution. After stirring for 4.5 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=9:1, v/v) to give 62.2 mg (Yield 59.0%) of the title compound.

m.p. 59-60° C.

¹H NMR (500 MHz, CDCl₃): δ ppm 7.62 (s, 1H), 7.23-7.29 (m, 3H), 7.00-7.03 (m, 4H), 6.67 (s, 1H), 6.55 (s, 1H), 4.57 (s, 1H), 4.09 (m, 4H), 3.95 (q, 2H), 3.72 (m, 1H), 3.59 (m, 1H), 3.17 (m, 1H), 2.85 (m, 1H), 1.45 (t, 3H), 1.36 (t, 3H)

EI MS, m/e=405

### EXAMPLE 80

Synthesis of 1-[(1H-imidazol-1-yl)carbonyl]-4,4-diphenyl-piperidine

1,1′-Carbonyldiimidazole (40 mg, 0.25 mmol) and 4,4-diphenyl-piperidine (49 mg, 0.25 mmol) were mixed in anhydrous THF (3 ml), and then DBU (84.3 μl, 0.56 mmol) was added dropwise to this reaction solution. After stirring for 4 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 48 mg (Yield 58%) of the title compound.

m.p.: 179-182° C.

¹H NMR (300 MHz, CDCl₃): δ ppm 7.86 (s, 1H), 7.67 (s, 1H), 7.09-7.34 (m, 11H), 3.67 (t, 3H, J=5.49 Hz), 2.50 (t, 3H, J=5.49 Hz)

### EXAMPLE 81

Synthesis of 1-[(1H-imidazol-1-yl)thiocarbonyl]-4,4-diphenyl-piperidine

1,1′-Thiocarbonyldiimidazole (45 mg, 0.25 mmol) and 4,4-diphenyl-piperidine (49 mg, 0.25 mmol) were mixed in anhydrous THF (3 ml), and then DBU (84.3 μl, 0.56 mmol) was added dropwise to this reaction solution. After stirring for 4 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:ethyl acetate:hexane=1:1, v/v) to give 56 mg (Yield 64.5%) of the title compound.

m.p.: 161-165° C.

¹H NMR (300 MHz, CDCl₃): δ ppm 7.84 (s, 1H), 7.08-7.35 (m, 12H), 3.97 (s, 3H), 2.50 (s, 3H)

### EXAMPLE 82

Synthesis of 1-[(1H-imidazol-1-yl)carbonyl]-4-ethoxycarbonyl-4-phenyl-piperidine

1-[(1H-imidazol-1-yl)carbonyl]-4-hydroxycarbonyl-4-phenyl-piperidine (50 mg, 0.17 mmol) and 95% sulfuric acid (10 μl, 0.17 mmol) were mixed in absolute ethanol (5 ml), and stirred under reflux for 5 hours. The reaction solution was cooled to room temperature and concentrated under reduced pressure. The residue was separated by column chromatography (eluent:methylene chloride:methanol=30:1, v/v) to give 12.2 mg (Yield 21.9%) of the title compound.

¹H NMR (300 MHz, CDCl₃): δ ppm 7.87 (s, 1H), 7.27-7.37 (m, 5H), 7.19 (s, 1H), 7.10 (s, 1H), 4.17 (q, 2H), 4.05 (m, 2H), 3.32 (m, 2H), 2.67 (m, 2H), 2.03 (m, 2H), 1.18 (t, 3H)

### EXAMPLE 83

Synthesis of 1-[(1H-imidazol-1-yl)carbonyl]-4-[(methoxycarbonyl)-(4-methoxy phenyl)-methyl]-piperazine

1,1′-Carbonyldiimidazole (20 mg, 0.13 mmol) and 4-[(methoxycarbonyl)-(4-methoxy phenyl)-methyl]-piperazine (35 mg, 0.13 mmol) were mixed in anhydrous THF (5 ml), and then DBU (32 μl, 0.21 mmol) was added dropwise to this reaction solution. After stirring for 12 hours at room temperature, the product was extracted with methylene chloride (30 ml×3). The organic solution was washed with aqueous sodium chloride solution and water, dried over anhydrous magnesium sulfate, and concentrated under reduced pressure. The residue was separated by column chromatography (eluent:methylene chloride:methanol=30:1, v/v) to give 37 mg (Yield 79.2%) of the title compound.

¹H NMR (300 MHz, CDCl₃): δ ppm 7.85, 7.18, 7.09 (3s, 3H), 7.24, 6.87 (2d, 4H, J=9 Hz), 3.79, 3.65 (2s, 6H), 3.79, 3.58, 3.19, 2.64, 2.58 (5m, 9H)

### EXAMPLE 84

Synthesis of 1-[(1H-imidazol-1-yl)carbonyl]-4-(diphenylmethyl)piperazine

1,1′-Carbonyldiimidazole (41 mg, 0.25 mmol) and 1-(diphenylmethyl)piperazine (64 mg, 0.25 mmol) were mixed in anhydrous THF (10 ml), and then DBU (48 μl, 0.32 mmol) was added dropwise to this reaction solution. After stirring for 2 hours at room temperature, the product was extracted with ethyl acetate (50 ml×3). The organic solution was washed with aqueous sodium chloride solution and water, dried over anhydrous magnesium sulfate, and concentrated under reduced pressure. The residue was separated by column chromatography (eluent:methylene chloride:methanol=20:1, v/v) to give 65 mg (Yield 75.1%) of the title compound.

¹H NMR (300 MHz, CDCl₃): δ ppm 7.83, 7.22, 7.11 (3s, 3H), 7.40-7.27 (m, 10H), 4.28 (s, 1H), 3.60, 2.46 (2m, 8H)

### EXAMPLE 85

Synthesis of 5-[(1H-imidazol-1-yl)carbonyl]-10,11-dihydro-5H-dibenzazepine

1,1′-Carbonyldiimidazole (415 mg, 2.5 mmol) and 10,11-dihydro-5H-dibenzazepine (500 mg, 2.5 mmol) were mixed in anhydrous THF (10 ml), and then 95% NaH (415 mg, 2.5 mmol) was added dropwise to this reaction solution. After stirring under reflux for 19 hours at 80° C., the product was extracted with diethylether (100 ml×3). The organic solution was washed with aqueous sodium chloride solution and water, dried over anhydrous magnesium sulfate, and concentrated under reduced pressure. The residue was separated by column chromatography (eluent:methylene chloride:methanol=5:1, v/v) to give 22 mg (Yield 3.0%) of the title compound.

m.p.: 189-190° C.

¹H NMR (300 MHz, CDCl₃): δ ppm 7.64 (s, 1H), 7.19-7.28 (m, 8H), 6.95 (s, 1H), 6.87 (s, 1H), 3.54 (br-m, 2H), 3.03 (br-m, 2H)

### EXAMPLE 86

Synthesis of 4-[(1H-imidazol-1-yl)carbonyl]-1,2,3,5-tetrahydro-4H-benzo[1,4]diazepine

2,3,4,5-Tetrahydro-1H-benzo[1,4]diazepine (238.7 mg, 1.6 mmol) and 95% NaH (44.8 mg, 1.77 mmol) were mixed in anhydrous THF (8 ml) and stirred for 30 minutes at room temperature. To this reaction solution was added dropwise 1,1′-carbonyldiimidazole (259.2 mg, 1.6 mmol), and the resulting mixture was stirred for 2.5 hours at room temperature. The product was extracted with diethylether (50 ml×3). The organic solution was washed with aqueous sodium chloride solution and water, dried over anhydrous magnesium sulfate, and concentrated under reduced pressure. The residue was separated by column chromatography (eluent:methylene chloride:methanol=100:1, v/v) to give 65.8 mg (Yield 17.0%) of the title compound.

m.p. 98-101° C.

¹H NMR (500 MHz, CDCl₃): δ ppm 6.64-7.15 (m, 7H), 4.34 (s, 2H), 4.29 (m, 2H), 3.43 (m, 2H)

### EXAMPLE 87

Synthesis of 6-[(1H-imidazol-1-yl)carbonyl]-8-phenyl-4,5,7,8-tetrahydro-6H-thieno[2,3]azepine

8-Phenyl-4,5,7,8-tetrahydro-6H-thieno[2,3]azepine (46.1 mg, 0.2 mmol) and 95% NaH (14.4 mg, 0.6 mmol) were mixed in anhydrous THF (1 ml) and stirred for 30 minutes at room temperature. To this reaction solution was added dropwise 1,1′-carbonyldiimidazole (32.6 mg, 0.2 mmol), and the resulting mixture was stirred for 1 hour at room temperature. The product was extracted with methylene chloride (50 ml×4). The organic solution was washed with aqueous sodium chloride solution and water, dried over anhydrous magnesium sulfate, and concentrated under reduced pressure. The residue was separated by column chromatography (eluent:methylene chloride:methanol=30:1, v/v) to give 30.7 mg (Yield 47.5%) of the title compound.

m.p. 152-154° C.

¹H NMR (500 MHz, CDCl₃): δ ppm 7.54 (s, 1H), 6.82-7.29 (m, 9H), 4.58 (m, 1H), 4.17 (dd, 1H, J=14.1, J=7.32 Hz), 4.06 (dd, 11, J=14.1, J=3.48 Hz), 3.79 (m, 1H), 3.71 (m, 1H), 3.26 (m, 1H), 3.05 (m, 1H)

Experiment 1: Anti-cancer activity

The anti-cancer activity and toxicity of the compound according to the present invention were evaluated in vitro using A549 (lung cancer), SUN638 (gastric cancer), HCT116 (rectal cancer), and A431 (ovarian cancer) cell lines. The above four (4) cell lines were purchased from Cancer Research Institute, Seoul National University College of Medicine. The compounds prepared in the Examples according to the present invention were used as the test compound, and the known cisplatin was used as the control compound. The experiment was basically carried out according to the process described in Monks, A., et al, Journal of National Cancer Institute. 83: 757-766 (1991) and the results are represented in the following Tables 2 and 3.

TABLE 2

| Compound | A549 Cell Line (μg/ml) | | | SUN638 Cell Line (μg/ml) | | |
|---|---|---|---|---|---|---|
| No. | GI₅₀ | TGI₅₀ | LC₅₀ | GI₅₀ | TGI₅₀ | LC₅₀ |
| 1 | 1.21 | 39.81 | >100 | 1.99 | 7.07 | >100 |
| 3 | 1.31 | 5.62 | >100 | 1.47 | 9.33 | >100 |
| 4 | 1.20 | 100 | >100 | 1.86 | 6.76 | >100 |
| 5 | 2.08 | 46.98 | >100 | 1.69 | 6.16 | >100 |
| 6 | 2.69 | 100 | >100 | 1.90 | 6.91 | >100 |
| 7 | 3.38 | 100 | >100 | 2.45 | 7.41 | >100 |
| 16 | 1.02 | 63.09 | >100 | 0.15 | 35.48 | >100 |
| 17 | 6.45 | 83.17 | >100 | 0.63 | 39.81 | >100 |
| 26 | 1.36 | 100 | >100 | 2.51 | 95.49 | >100 |
| 28 | 1.31 | 34.67 | >100 | 1.99 | 7.07 | >100 |
| 38 | 1.28 | 4.36 | >100 | 2.75 | 100 | >100 |
| 48 | 1.07 | 72.44 | >100 | 1.62 | 35.48 | >100 |
| 53 | 2.75 | 8.91 | >100 | 2.18 | 5.49 | >100 |
| 55 | 1.77 | 25.70 | >100 | 1.09 | 6.91 | >100 |
| 56 | 2.04 | 12.30 | >100 | 1.34 | 6.62 | >100 |
| 57 | 1.77 | 15.13 | >100 | 1.69 | 6.91 | >100 |
| 58 | 2.75 | 100 | >100 | 2.39 | 7.76 | >100 |
| 61 | 6.02 | 32.35 | >100 | 2.95 | 6.91 | >100 |
| 62 | 0.48 | 42.65 | >100 | 0.61 | 28.18 | >100 |
| 63 | 2.23 | 4.89 | >100 | 1.81 | 5.12 | >100 |
| 66 | 0.38 | 54.95 | >100 | 0.21 | 1.73 | >100 |
| 67 | 0.66 | 17.78 | >100 | 1.51 | 8.12 | >100 |
| 68 | 0.23 | 100 | >100 | 0.22 | 97.72 | >100 |
| 69 | 0.36 | 97.72 | >100 | 0.13 | 3.63 | >100 |
| 70 | 1.12 | 100 | >100 | 1.78 | 7.24 | >100 |
| 71 | 4.78 | 93.32 | >100 | 2.63 | 9.12 | >100 |
| 72 | 2.23 | 100 | >100 | 1.44 | 7.24 | >100 |
| 80 | 1.38 | 100 | >100 | 1.62 | 5.88 | >100 |
| Cisplatin | 3.8 | 47.3 | >100 | 1.7 | 13.3 | >100 |

As seen from the result in Table 2 above, the compounds of the present invention show a superior anti-cancer activity in A549 (lung cancer) and SUN638 (gastric cancer) cell lines to the control compound. More specifically, the anti-cancer activity of the compounds of the present invention is as high as or up to 10 times higher than that of the control compound in A549 and SUN638 cell lines.

TABLE 3

| Compound | A431 Cell Line (μg/ml) | | | HCT116 Cell Line (μg/ml) | | |
|---|---|---|---|---|---|---|
| No. | GI₅₀ | TGI₅₀ | LC₅₀ | GI₅₀ | TGI₅₀ | LC₅₀ |
| 3 | 2.04 | 26.30 | >100 | 1.31 | 5.62 | >100 |
| 4 | — | — | — | 0.12 | 3.54 | >100 |
| 5 | — | — | — | 0.15 | 4.07 | >100 |
| 6 | — | — | — | 0.39 | 3.31 | >100 |
| 7 | — | — | — | 0.15 | 4.08 | >100 |
| 38 | 1.41 | 7.24 | >100 | 1.28 | 4.36 | >100 |
| 48 | 1.65 | 100 | >100 | 0.67 | 17.38 | >100 |
| 55 | 1.38 | 67.60 | >100 | 2.08 | 13.48 | >100 |
| 56 | 1.31 | 28.84 | >100 | 1.47 | 6.02 | >100 |
| 57 | 1.51 | 8.31 | >100 | 1.34 | 4.89 | >100 |
| 58 | — | — | — | 0.66 | 12.88 | >100 |
| 62 | — | — | — | 0.38 | 19.05 | >100 |
| 63 | — | — | — | 1.77 | 4.78 | >100 |
| 66 | 0.28 | 100 | >100 | 0.19 | 7.76 | >100 |
| 67 | 0.70 | 30.19 | >100 | 0.23 | 13.48 | >100 |
| 68 | — | — | — | 0.24 | 10.96 | >100 |
| 69 | — | — | — | 0.13 | 10.71 | >100 |
| 70 | — | — | — | 1.31 | 23.98 | >100 |
| 71 | — | — | — | 2.69 | 14.12 | >100 |
| 72 | — | — | — | 0.10 | 9.42 | >100 |
| 74 | — | — | — | 1.44 | 7.24 | >100 |
| 80 | — | — | — | 1.47 | 16.88 | >100 |
| Cisplatin | 3.2 | 28.7 | >100 | 2.9 | 32.77 | >100 |

Judging from the result in Table 3 above, the compounds of the present invention also show a superior anti-cancer activity in HCT116 (rectal cancer) and A431 (ovarian cancer) cell lines to the control compound. Specifically, the compounds of the present invention exhibit the same or a maximum of about 30 times higher anti-cancer activity than the control compound in HCT1 16 cell line. In A431 cell line, most of the compounds according to the present invention show twice or more higher anti-cancer activity than cisplatin and some compounds show five to eleven times higher activity than the control compound.

### Experiment 2

Acute toxicity test

The test for acute toxicity was carried out according to Notice No. 1999-61 of KFDA (‘Standard for Toxicity Test of Medicines’) and to Notice No. 1998-17 of KFDA (‘Standard for Safety Control of Medicines’) as follows.

1. Test System

(1) Test Animal: SPF ICR mouse

(2) Age of the Test Animal: 4 weeks from acquisition 5 weeks from the start of administration

(3) Gender and Number of Test Animal: respective 18 he and she-mice (6 groups in each of he and she-mice, 3 mice per group)

(4) Breeding Environment: Temperature 22±3° C., Relative humidity ±10%, Illumination 150-300 Lux

(5) Method of Administration: Single administration per oral

(6) Items to be Observed: General symptoms, appearance, weight and autopsy of dead mice

(7) Test Compound: Compounds prepared in Examples 2, 38, 57, 61, 62, 66 and 70

(8) Medium: 0.5% Tween 80

2. Test Result

As a result of the 7-day observation after the test compound was administered, no animals were dead in any test group. The Minimal Lethal Dose of single administration per oral in mouse was determined to be 354.3 mg/kg to 2000 mg/kg or more in both he and she-mice (see, Table 4). This value is still higher than those of various drugs which are clinically used at the present time, and thus, the compound according to the present invention proved very safe.

TABLE 4

| Test Compound | LD₅₀ (mg/kg) |
|---|---|
| Cisplatin | 9.7 |
| Carboplatin | 150 |
| Doxorubicin | 21.1 |
| Compound of Example 2 | 2000 (male and female) |
| Compound of Example 38 | 2000 (male and female) |
| Compound of Example 57 | 1156.5 (male and female) |
| Compound of Example 61 | 2331.7 (male) |
| | 1143 (female) |
| Compound of Example 62 | 354.3 (male) |
| | 465.5 (female) |
| Compound of Example 66 | 1921 (male) |
| | 2000 (female) |
| Compound of Example 70 | 2000 (male and female) |

1. A compound represented by the following formula (I): 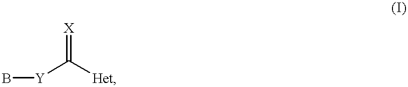
, its pharmaceutically acceptable acid addition salt or stereoisomer, in which
X represents O or S, or represents imino substituted or unsubstituted by cyano,
Y represents a direct bond, NH, O or S,
B represents C₁-C₈-alkyl, or represents a radical having one of the following formulas: 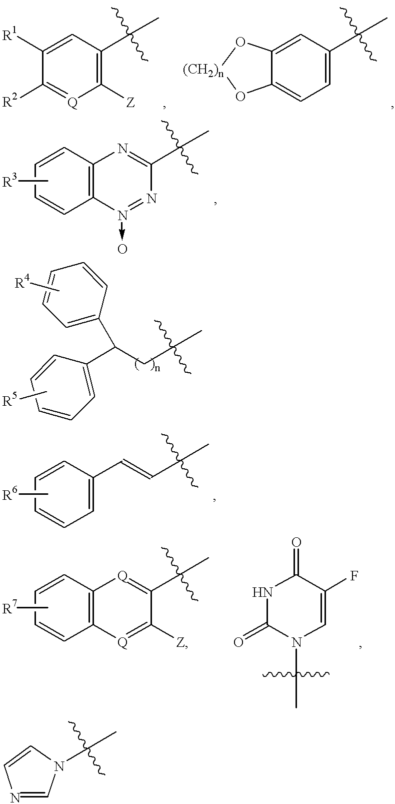
wherein
R¹ and R² independently of one another represent hydrogen, C₁-C₈-alkyl or cyano, or represent amidino substituted or unsubstituted by C₁-C₈-alkyl,
Q represents CH or N,
Z represents C₁-C₄-alkoxy or phenoxy,
n represents an integer of 0 to 3,
R³, R⁴, R⁵, R⁶ and R⁷ independently of one another represent hydrogen, C₁-C₈-alkyl or halogen,
Het represents a radical having one of the following formulas: 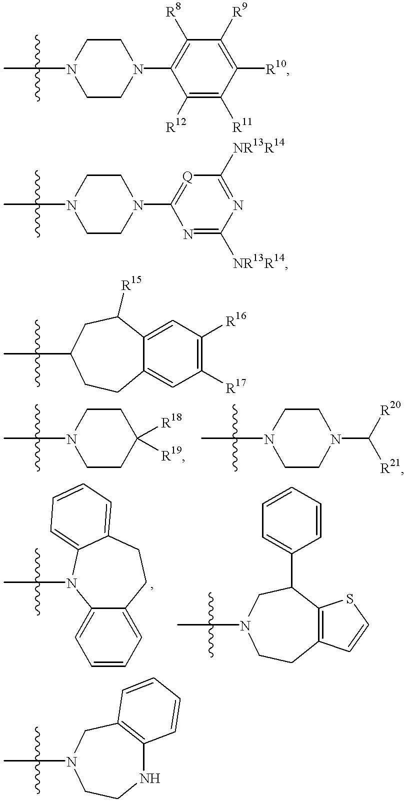
wherein
R⁸, R⁹, R¹⁰, R¹¹ and R¹² independently of one another represent hydrogen, C₁-C₈-alkyl, C₁-C₈-alkoxy or halogen,
R¹³ and R¹⁴ independently of one another represent hydrogen, C₁-C₈-alkyl, C₁-C₈-alkoxy, C₂-C₅-alkenyl, C₃-C₆-cycloalkyl or C₃-C₆-cycloalkyl-C₁-C₄- alkyl, or R¹³ and R¹⁴ together with the nitrogen atom to which they are attached represent pyrrolinyl or pyrrolidinyl,
R¹⁵ represents hydrogen, or represents phenyl or benzyl each of which is substituted or unsubstituted by 1 to 5 identical or different halogen atoms,
R¹⁶ and R¹⁷ independently of one another represent hydrogen, C₁-C₄-alkyl, C₁-C₄-alkoxy or hydroxy,
R¹⁸ and R¹⁹ independently of one another represent hydrogen, hydroxycarbonyl or C₁-C₄-alkoxycarbonyl, or represent phenyl substituted or unsubstituted by 1 to 5 identical or different halogen atoms or C₁-C₄-alkoxy, or together represent diphenylmethylene or benzolactone of formula 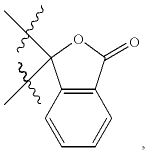
and
R²⁰ and R²¹ independently of one another represent hydrogen, or represent phenyl substituted or unsubstituted by 1 to 5 identical or different halogen atoms, C₁-C₈-alkyl or C₁-C₄-alkoxy, or represent C₁-C₄-alkoxycarbonyl.

2. The compound of claim 1, wherein the compound is selected from the group consisting of the following:
3-[N-(5,6-dimethyl-2-methoxypyridin-3-yl)aminocarbonyl]-7,8-dimethoxy-1-phenyl-2,3,4,5-tetrahydro-3H-benzazepine;
3-[N-(2-methoxyquinoxalin-3-yl)aminocarbonyl]-7,8-dimethoxy-1-phenyl-2,3,4,5-tetrahydro-3H-benzazepine;
3-[N-(2-methoxyquinoxalin-3-yl)aminocarbonyl]-(S)-7,8-dimethoxy-1-phenyl-2,3,4,5-tetrahydro-3H-benzazepine;
3-[N-(2-methoxyquinoxalin-3-yl)aminocarbonyl]-(R)-7,8-dimethoxy-1-phenyl-2,3,4,5-tetrahydro-3H-benzazepine;
3-[N-(2-methoxyquinoxalin-3-yl)aminocarbonyl]-7,8-dimethoxy-1-(4-fluoro) phenyl-2,3,4,5-tetrahydro-3H-benzazepine;
3-[N-(2-methoxyquinoxalin-3-yl)aminocarbonyl]-7,8-dimethoxy-1-(4-chloro) phenyl-2,3,4,5-tetrahydro-3H-benzazepine;
3-[N-(2-ethoxyquinoxalin-3-yl)aminocarbonyl]-7,8-dimethoxy-1-phenyl-2,3,4,5-tetrahydro-3H-benzazepine;
3-[N-(2-phenoxyquinoxalin-3-yl)aminocarbonyl]-7,8-dimethoxy-1-phenyl-2,3,4,5-tetrahydro-3H-benzazepine;
3-[N-(diphenylmethyl)aminocarbonyl]-7,8-dimethoxy-1-phenyl-2,3,4,5-tetrahydro-3H-benzazepine;
3-[N-(diphenylmethyl)aminocarbonyl]-7,8-dimethoxy-1-(4-fluoro)phenyl-2,3,4,5-tetrahydro-3H-benzazepine;
3-[N-(diphenylmethyl)aminocarbonyl]-7,8-dimethoxy-1-(4-chloro)phenyl-2,3,4,5-tetrahydro-3H-benzazepine;
1-[N-(5,6-dimethyl-2-methoxypyridin-3-yl)aminocarbonyl]-4-diphenylmethylene-piperidine;
1-[N-(diphenylmethyl)aminocarbonyl]-4-diphenylmethylene-piperidine;
1-[N-(2,2-diphenylethan-1-yl)aminocarbonyl]-4-diphenylmethylene-piperidine;
1-[N-(5,6-dimethyl-2-methoxypyridin-3-yl)aminocarbonyl]-4,4-diphenyl-piperidine;
1-[N-(2-methoxyquinoxalin-3-yl)aminocarbonyl]-4,4-diphenyl-piperidine;
1-[N-(diphenylmethyl)aminocarbonyl]-4,4-diphenyl-piperidine;
1-[N-(diphenylmethyl)aminocarbonyl]-4-hydroxycarbonyl-4-phenyl-piperidine;
1-[N-(5-ethyl-6-methyl-2-methoxypyridin-3-yl)aminocarbonyl]-4-hydroxycarbonyl-4-phenyl-piperidine;
1 -[N-(2,2-diphenylethan- 1-yl)aminocarbonyl]-4-hydroxycarbonyl-4-phenyl-piperidine;
1-[N-(5-ethyl-6-methyl-2-methoxypyridin-3-yl)aminocarbonyl]spiro[isobenzofuran-1(3H),4-piperidin]-3-one;
1-[N-(2-methoxyquinoxalin-3-yl)aminocarbonyl]spiro[isobenzofuran-1(3H),4-piperidin]-3-one;
1-[(5,6-dimethyl-2-methoxypyridin-3-yl)aminocarbonyl]-4-(diphenylmethyl) piperazine;
1-[(5,6-dimethyl-2-methoxypyridin-3-yl)amino-N-cyanocarboimidate]-4-(3,5-dimethylphenyl)piperazine;
1-[(5,6-dimethyl-2-methoxypyridin-3-yl)aminocarbonyl]-4-[4,6-bis(propylamino)-1,3,5-triazin-2-yl]piperazine;
1-[(2-methoxy-5-cyanophenyl)aminocarbonyl]-4-(3,5-dimethylphenyl)piperazine;
1-[(1,4-benzodioxan-6-yl)amino-N-cyanocarboimidate]-4-(3,5-dimethylphenyl) piperazine;
1-[(benzo-1,2,4-triazin-1-N-oxide-3-yl)aminocarbonyl]-4-(3,5-dimethylphenyl) piperazine;
1-[(benzo-1,2,4-triazin-1-N-oxide-3-yl)aminocarbonyl]-4-(diphenylmethyl) piperazine;
1-[(2-phenylvinyl)aminocarbonyl]-4-(3,5-dimethylphenyl)piperazine;
1-[(diphenylmethyl)aminocarbonyl]-4-(diphenylmethyl)piperazine;
1-[(diphenylmethyl)aminocarbonyl]-4-(3,5-dimethylphenyl)piperazine;
1-[(diphenylmethyl)aminothiocarbonyl]-4-(diphenylmethyl)piperazine;
1-[(diphenylmethyl)aminocarbonyl]-4-[4,6-bis(propylamino)-1,3,5-triazin-2-yl]piperazine;
1-[(diphenylmethyl)amino-N-cyanocarboimidate]-4-(3,5-dimethylphenyl) piperazine;
1-[(2,2-diphenylethan-1-yl)aminocarbonyl]-4-(3,5-dimethylphenyl)piperazine;
1-[(2,2-diphenylethan-1-yl)aminocarbonyl]-4-(diphenylmethyl)piperazine;
1-[(diphenylmethyl)aminocarbonyl]-4-(4,6-bis-allylamino-[1,3,5]triazin-2-yl) piperazine;
1-[(diphenylmethyl)aminocarbonyl]-4-{4,6-bis-(allyl-cyclohexylamino)-[1,3,5]triazin-2-yl}piperazine;
1-[(diphenylmethyl)aminocarbonyl]-4-{4,6-bis-[ethyl-(2-methylallyl)amino]-[1,3,5]triazin-2-yl}piperazine;
1-[(diphenylmethyl)aminocarbonyl]-4-(4,6-bis-diallylamino-[1,3,5]triazin-2-yl)piperazine;
1-[(diphenylmethyl)aminocarbonyl]-4-(4,6-bis-cyclopropylmethylamino-[1,3,5]triazin-2-yl)piperazine;
1-[(2,2-diphenylethan-1-yl)amino-N-cyanocarboimidate]-4-(4,6-bis-allylamino-[1,3,5]triazin-2-yl)piperazine;
1-[(diphenylmethyl)amino-N-cyanocarboimidate]-4-(4,6-bis-allylamino-[1,3,5]triazin-2-yl)piperazine;
1-[(2,2-diphenylethan-1-yl)aminocarbonyl]-4-(4,6-bis-allylamino-[1,3,5]triazin-2-yl)piperazine;
1-[5-ethyl-6-methyl-2-methoxypyridin-3-yl)aminocarbonyl]-4-(4,6-bis-allylamino-[1,3,5]triazin-2-yl)piperazine;
1-[(5-ethyl-6-methyl-2-methoxypyridin-3-yl)aminocarbonyl]-4-[(2,6-dipyrrolidin-1-yl)pyrimidin-4-yl]piperazine;
1-[N-(2-methoxyquinoxalin-3-yl)aminocarbonyl]-4-(diphenylmethyl)piperazine;
1-[N-(2-ethoxyquinoxalin-3-yl)aminocarbonyl]-4-(diphenylmethyl)piperazine;
1-(2-phenoxyquinoxalin-3-yl)aminocarbonyl]-4-(diphenylmethyl)piperazine;
1-[N-(2-methoxyquinoxalin-3-yl)aminocarbonyl]-4-(4,6-bis-allylamino-[1,3,5]triazin-2-yl)piperazine;
1-[N-(2-methoxyquinoxalin-3-yl)aminocarbonyl]-4-{4,6-bis-[ethyl-(2-methylallyl)amino]-[1,3,5]triazin-2-yl}piperazine;
1-[N-(2-methoxyquinoxalin-3-yl)aminocarbonyl]-4-(4,6-bis-diallylamino-[1,3,5]triazin-2-yl)piperazine;
1-[(5-fluoro-1H-pyrimidin-2,4-dioxo-1-yl)carbonyl]-4-(3,5-dimethylphenyl) piperazine;
1-[(1H-imidazol-1-yl)carbonyl]-4-(3,5-dimethylphenyl)piperazine;
1-[(1H-imidazol-1-yl)thiocarbonyl]-4-(3,5-dimethylphenyl)piperazine;
1-[(1H-imidazol-1-yl)carbonyl]-4-(3,5-dimethylphenyl)piperazine hydrochloride
1-[(1H-imidazol-1-yl)carbonyl]-4-(3,5-dimethoxyphenyl)piperazine;
1-[(1H-imidazol-1-yl)carbonyl]-4-(4,6-bis-allylamino-[1,3,5]triazin-2-yl) piperazine;
1-[(1H-imidazol-1-yl)thiocarbonyl]-4-(4,6-bis-allylamino-[1,3,5]triazin-2-yl) piperazine;
1-[(1H-imidazol-1-yl)carbonyl]-4-(4,6-bis-diallylamino-[1,3,5]triazin-2-yl) piperazine;
1-[(1H-imidazol-1-yl)carbonyl]-4-(4,6-bis-cyclopropylmethylamino-[1,3,5]triazin-2-yl)piperazine
1-[(1H-imidazol-1-yl)carbonyl]-4-{4,6-bis-[ethyl-(2-methylallyl)amino]-[1,3,5]triazin-2-yl}piperazine;
1-[(1H-imidazol-1-yl)carbonyl]-4-[4,6-bis-(allyl-cyclohexyl-amino)-[1,3,5]triazin-2-yl]piperazine;
1-[(1H-imidazol-1-yl)carbonyl]-4-{4,6-bis-(2,5-dihydropyrrol-1-yl)-[1,3,5]triazin-2-yl}piperazine;
3-[(1H-imidazol-1-yl)carbonyl]-7,8-dimethoxy-1-phenyl-2,3,4,5-tetrahydro-3H-benzazepine;
3-[(1H-imidazol-1-yl)thiocarbonyl]-7,8-dimethoxy-1-phenyl-2,3,4,5-tetrahydro-3H-benzazepine;
3-[(1H-imidazol-1-yl)carbonyl]-(R)-7,8-dimethoxy-1-phenyl-2,3,4,5-tetrahydro-3H-benzazepine;
3-[(1H-imidazol-1-yl)carbonyl]-(S)-7,8-dimethoxy-1-phenyl-2,3,4,5-tetrahydro-3H-benzazepine;
3-[(1H-imidazol-1-yl)carbonyl]-7,8-dimethoxy-1-(4-fluoro)phenyl-2,3,4,5-tetrahydro-3H-benzazepine;
3-[(1H-imidazol-1-yl)carbonyl]-7,8-dimethoxy-1-(4-chloro)phenyl-2,3,4,5-tetrahydro-3H-benzazepine;
3-[(1H-imidazol-1-yl)carbonyl]-8-methoxy-1-phenyl-2,3,4,5-tetrahydro-3H-benzazepine;
3-[(1H-imidazol-1-yl)carbonyl]-8-methoxy-1-(4-fluoro)phenyl-2,3,4,5-tetrahydro-3H-benzazepine;
3-[(1H-imidazol-1-yl)carbonyl]-8-methoxy-1-(4-chloro)phenyl-2,3,4,5-tetrahydro-3H-benzazepine;
3-[(1H-imidazol-1-yl)carbonyl]-1-phenyl-2,3,4,5-tetrahydro-3H-benzazepine;
3-[(1H-imidazol-1-yl)carbonyl]-1-(4-fluoro)phenyl-2,3,4,5-tetrahydro-3H-benzazepine;
3-[(1H-imidazol-1-yl)carbonyl]-1-(4-chloro)phenyl-2,3,4,5-tetrahydro-3H-benzazepine;
3-[(1H-imidazol-1-yl)carbonyl]-7,8-dihydroxy-1-phenyl-2,3,4,5-tetrahydro-3H-benzazepine;
3-[(1H-imidazol-1-yl)carbonyl]-7,8-diethoxy-1-phenyl-2,3,4,5-tetrahydro-3H-benzazepine;
1-[(1H-imidazol-1-yl)carbonyl]-4,4-diphenyl-piperidine;
1-[(1H-imidazol-1-yl)thiocarbonyl]-4,4-diphenyl-piperidine;
1-[(1H-imidazol-1-yl)carbonyl]-4-ethoxycarbonyl-4-phenyl-piperidine;
1-[(1H-imidazol-1-yl)carbonyl]-4-[(methoxycarbonyl)-(4-methoxyphenyl)methyl]-piperazine;
1-[(1H-imidazol-1-yl)carbonyl]-4-(diphenylmethyl)piperazine;
5-[(1H-imidazol-1-yl)carbonyl]-10,11-dihydro-5H-dibenzazepine;
5-[(1H-imidazol-1-yl)carbonyl]-1,2,3,5-tetrahydro-4H-benzo[1,4]diazepine; and
6-[(1H-imidazol-1-yl)carbonyl]-8-phenyl-4,5,7,8-tetrahydro-6H-thieno[2,3]azepine.

3. A process for preparing the compound of formula (I) defined in claim 1, characterized in that
(a) a compound represented by the following formula (II):
B—YH (II)
wherein B and Y are as defined in claim 1, and a compound represented by the following formula (III): 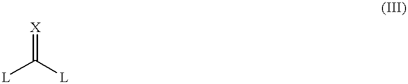
wherein X is as defined in claim 1, and L represents a leaving group, are reacted in a solvent in the presence of a base with a compound represented by the following formula (IV):
H—Het (IV)
wherein Het is as defined in claim 1, to produce the compound of formula (I); or
(b) a compound represented by the following formula (V): 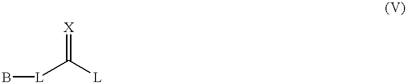
wherein B, Y and X are as defined in claim 1 and L are as defined above, is reacted in a solvent in the presence of a base with the compound of formula (IV) to produce the compound of formula (I); or
(c) a compound represented by the following formula (VI): 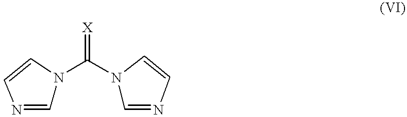
wherein X is as defined in claim 1, is reacted in a solvent in the presence of a base with the compound of formula (IV) to produce a compound represented by the following formula (Ida): 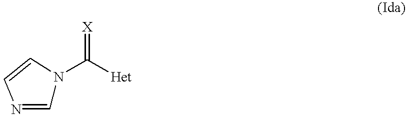
wherein X and Het are as defined in claim 1, or optionally deprotection, alkylation or esterification reaction is further carried out.

4. An anti-cancer composition comprising as an active ingredient the compound of formula (I), its pharmaceutically acceptable acid addition salt or stereoisomer as defined in claim 1, together with a pharmaceutically acceptable carrier.
